(19) United States
(12) Patent Application Publication
(10) Pub. No.: US 2003/0045468 A1
(43) Pub. Date: Mar. 6, 2003

(54) Compositions and methods for the therapy, diagnosis and monitoring of breast cancer

(75) Inventors: Steven P. Fling, Bainbridge Island, WA (US); Teresa M. Foy, Federal Way, WA (US); Jonathan D. Clapper, Seattle, WA (US); Aijun Wang, Issaquah, WA (US); Jeffrey C. Johnson, Des Moines, WA (US); Patricia D. McNeill, Federal Way, WA (US); R. Alec Sutherland, Bothell, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 10/042,945

(22) Filed: Jan. 8, 2002

Related U.S. Application Data
(63) Continuation-in-part of application No. 10/008,045, filed on Dec. 8, 2001, now abandoned.
Application No. 10/008,045 is a continuation-in-part of application No. 09/757,417, filed on Jan. 8, 2001.
Application No. 09/757,417 is a continuation-in-part of application No. 09/580,376, filed on May 26, 2000.
(60) Provisional application No. 60/137,048, filed on Jun. 1, 1999.
(60) Provisional application No. 60/136,528, filed on May 28, 1999.

(51) Int. Cl. A61K 39/395; G01N 33/574; C07H 21/04; C07K 16/30

(52) U.S. Cl. 514/12; 536/23.5; 435/7.23; 530/387.1; 424/155.1

(57) ABSTRACT

Compositions and methods for the therapy, diagnosis and monitoring of breast cancer are disclosed. Compositions may comprise one or more mammaglobin epitopes, or antibodies or T cells thereto, and may be used, for example, for the prevention and treatment of breast cancer. Diagnostic methods based on detecting the presence of mammaglobin epitopes, or antibodies or T cells thereto, in a sample are also provided. Also provided are methods for detecting RNA encoding mammaglobin in patient blood or fractions thereof. These methods may be used to detect and/or monitor the progression of breast cancer.

### BACKGROUND OF THE INVENTION

1. Field of the Invention

The present invention relates generally to therapy, diagnosis and monitoring of cancer, such as breast cancer. The invention is more specifically related to specific epitopes of mammaglobin, to antibodies and immune cells that recognize such epitopes and to methods for detecting mammaglobin in patient serum. Such peptides, antibodies and cells may be used in vaccines and pharmaceutical compositions for prevention and treatment of breast cancer, and for the diagnosis and monitoring of breast cancers.

2. Description of the Related Art

Breast cancer is a significant health problem for women in the United States and throughout the world. Although advances have been made in detection and treatment of the disease, breast cancer remains the second leading cause of cancer-related deaths in women, affecting more than 180,000 women in the United States each year. For women in North America, the life-time odds of getting breast cancer are now one in eight.

No vaccine or other universally successful method for the prevention or treatment of breast cancer is currently available. Management of the disease currently relies on a combination of early diagnosis (through routine breast screening procedures) and aggressive treatment, which may include one or more of a variety of treatments such as surgery, radiotherapy, chemotherapy and hormone therapy. The course of treatment for a particular breast cancer is often selected based on a variety of prognostic parameters, including an analysis of specific tumor markers. See, e.g., Porter-Jordan and Lippman, Breast Cancer 8:73-100, 1994. However, the use of established markers often leads to a result that is difficult to interpret, and the high mortality observed in breast cancer patients indicates that improvements are needed in the treatment, diagnosis and prevention of the disease.

In spite of considerable research into therapies and diagnostic methods, there is a need in the art for improved methods for detecting and treating breast cancers. The present invention fulfills these needs and further provides other related advantages.

### BRIEF SUMMARY OF THE INVENTION

In one aspect, the present invention provides polynucleotide compositions comprising a sequence selected from the group consisting of:

(a) sequences provided in SEQ ID NOs:19-26, 46, 51-55, and 69;

(b) complements of the sequences provided in SEQ ID NOs:19-26, 46, 51-55, and 69;

(c) sequences consisting of at least 20, 25, 30, 35, 40, 45, 50, 75 and 100 contiguous residues of a sequence provided in SEQ ID NOs:19-26, 46, 51-55, and 69;

(d) sequences that hybridize to a sequence provided in SEQ ID NOs:19-26, 46, 51-55, and 69, under moderate or highly stringent conditions;

(e) sequences having at least 75%, 80%, 85%, 90%, 95%, 96%, 97%, 98% or 99% identity to a sequence of SEQ ID NOs:19-26, 46, 51-55, and 69;

(f) degenerate variants of a sequence provided in SEQ ID NOs:19-26, 46, 51-55, and 69.

In one preferred embodiment, the polynucleotide compositions of the invention are expressed in at least about 20%, more preferably in at least about 30%, and most preferably in at least about 50% of breast tumors samples tested, at a level that is at least about 2-fold, preferably at least about 5-fold, and most preferably at least about 10-fold higher than that for normal tissues.

The present invention, in another aspect, provides polypeptide compositions comprising an amino acid sequence that is encoded by a polynucleotide sequence described above.

The present invention further provides polypeptide compositions comprising an amino acid sequence selected from the group consisting of sequences recited in SEQ ID NOs:1-7, 11-18, 27-45, 50, and 56-60.

In certain preferred embodiments, the polypeptides and/or polynucleotides of the present invention are immunogenic, i.e., they are capable of eliciting an immune response, particularly a humoral and/or cellular immune response, as further described herein.

The present invention further provides fragments, variants and/or derivatives of the disclosed polypeptide and/or polynucleotide sequences, wherein the fragments, variants and/or derivatives preferably have a level of immunogenic activity of at least about 50%, preferably at least about 70% and more preferably at least about 90% of the level of immunogenic activity of a polypeptide sequence set forth in SEQ ID NOs:1-7, 11-18, 27-45, 50, and 56-60 or a polypeptide sequence encoded by a polynucleotide sequence set forth in SEQ ID NOs:19-26, 46, 51-55, and 69.

The present invention further provides polynucleotides that encode a polypeptide described above, expression vectors comprising such polynucleotides and host cells transformed or transfected with such expression vectors.

Within other aspects, the present invention provides pharmaceutical compositions comprising a polypeptide or polynucleotide as described above and a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof, and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Illustrative antigen presenting cells include dendritic cells, macrophages, monocytes, fibroblasts and B cells.

Within related aspects, pharmaceutical compositions are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant. The fusions proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with breast cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with breast cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4⁺ and/or CD8⁺ T cells isolated from a patient with one or more of: (i) a polypeptide comprising at least an immunogenic portion of a polypeptide disclosed herein; (ii) a polynucleotide encoding such a polypeptide; and (iii) an antigen-presenting cell that expresses such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer, preferably a breast cancer, in a patient comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody.

The present invention also provides, within other aspects, methods for monitoring the progression of a cancer in a patient. Such methods comprise the steps of: (a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample, e.g., tumor sample, serum sample, etc., obtained from a patient with an oligonucleotide that hybridizes to a polynucleotide that encodes a polypeptide of the present invention; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a polypeptide of the present invention; (b) detecting in the sample an amount of a polynucleotide that hybridizes to the oligonucleotide; (c) repeating steps (a) and (b) using a biological sample obtained from the patient at a subsequent point in time, and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE SEVERAL VIEWS OF THE DRAWINGS

FIG. 1A is a summary of representative rabbit and mouse monoclonal antibodies raised against the human mammaglobin protein. Included is a summary of assays in which these anti-mammaglobin monoclonal antibodies have been used to detect mammaglobin. The epitope binding sequence for each monoclonal antibody (SEQ ID Nos: 11-18) is also listed. Abbreviations are: n.d.=not determined; FACS =fluorescence activate cell sorter; IHC=immunohistochemistry.

FIGS. 1B-1C present the CDR sequence for rabbit monoclonal antibodies 6A1 (SEQ ID NO: 19), 16D8 (SEQ ID Nos: 20-21), 6B12 (SEQ ID NO: 22), 2D3 (SEQ ID NO: 23), 14A12 (SEQ ID NO: 24), 29C11 (SEQ ID NO: 25) and 31A5 (SEQ ID NO: 26).

FIG. 2 presents the human mammaglobin amino acid sequence (SEQ ID NO: 27), along with peptide and recombinant regions used for epitope mapping studies. Various peptides (Pro 1-9 (SEQ ID NO: 27), Pro-20 (SEQ ID NO: 27) and Glob-2 (SEQ ID NO: 27)) spanning mammaglobin protein sequence were synthesized and used for epitope mapping of the monoclonal antibodies using the ELISA method. Each peptide sequence is indicated in bold and underlined. In addition, an N-terminal recombinant fragment of mammaglobin (SEQ ID NO: 28) was also used for epitope mapping studies.

FIGS. 3A-3D present epitope mapping data for the rabbit and mouse monoclonal antibodies obtained by the ELISA method. FIG. 3A shows the epitope binding regions of the mouse monoclonal antibodies. Shaded areas are considered positive for the antibody. Epitope binding specificity for the affinity-purified rabbit polyclonal 967 is also demonstrated. FIGS. 3B-3D present epitope mapping data for the rabbit monoclonal antibodies 6B12 (FIG. 3B), 29C11 (FIG. 3C) and 2D3 (FIG. 3D) using decreasing concentration of mammaglobin peptides and recombinant fragments.

FIGS. 4A-4B present the results of monoclonal antibody characterization by FACS analysis. Each monoclonal antibody was used to detect mammaglobin expression in MDA-MB-415 cells. Samples were fixed in 2% formaldehyde and permeabilized with 0.5% saponin. MCF-7 cells do not express mammaglobin and were used as a negative control.

FIG. 5 presents Western blot detection of mammaglobin by each monoclonal antibody. SDS-PAGE was performed on media in which MDA-MB-415 cells were grown, MDA-MB-415 cell lysate and bacterially expressed recombinant mammaglobin, as indicated. Mammaglobin expression was detected with the indicated antibody.

FIG. 6 is a table showing mammaglobin expression in breast tissue, but not in other tissues tested. Mammaglobin expression in various tissues was evaluated by immunohistochemistry analysis using a combination of 29C11 and 31A5 rabbit monoclonal antibodies.

FIGS. 7A-7C are graphs illustrating the results of sandwich assays performed using the indicated rabbit monoclonal antibodies to detect mammaglobin in lysates and supernatants of MB415 cells.

FIG. 8 is a graph showing the standard curve for a sandwich assay using the polyclonal anti-967 serum in combination with the monoclonal antibody 2D3 biotinylated.

FIG. 9 is a table showing the results of sandwich assays using the representative indicated antibodies to detect mammaglobin in patients with and without breast cancer.

FIG. 10 presents the human mammaglobin amino acid sequence (SEQ ID NO: 27), with underlined and bold peptide regions (SEQ ID Nos: 29-36) used for epitope mapping studies.

FIGS. 11A and 11B are graphs illustrating the recognition of CD4 T cell lines for mammaglobin and various portions thereof, as indicated. FIG. 11A shows T cell proliferation of three different CD4 T cell lines in response to various proteins and peptides. FIG. 11B shows interferon-y production by the same cells lines in response to the same proteins and peptides.

FIG. 12 presents the human mammaglobin amino acid sequence (SEQ ID NO: 27), along with peptide regions (SEQ ID Nos: 37-45) used for CD4⁺ and T cell epitope mapping studies.

FIGS. 13A-13C are graphs illustrating the recognition of JurkatA2 Kb cells pulsed with mgb-1 by CTL from HLA A2 transgenic mice immunized with mgb 1. CTL from three different mice were tested at different effector:target ratios, as indicated. Each figure shows the percent specific lysis of cells that are (solid circles) and are not (open circles) pulsed with mgb-1.

FIGS. 14A-14C are graphs illustrating the recognition of JurkatA2 Kb cells pulsed with mgb-1 (triangles) or expressing full length mammaglobin (mammaglobin) by CTL from HLA A2 transgenic mice immunized with mgb 1. CTL from three different mice were tested at different effector:target ratios, as indicated. In each figure, the percent specific lysis of cells that do not express mgb-1 or mammaglobin is represented by circles.

FIGS. 15A and 15B are a histogram showing the tissue distribution for mammaglobin. Copies of mammaglobin per ng β-actin are shown for a variety of normal and tumor tissues, as indicated.

FIG. 16 is a graph showing the number of copies of mammaglobin message in the breast cancer cell line MB415 as a function of the amount of cells.

FIG. 17 is a histogram showing the detection of mammaglobin in epithelial cells isolated, using the Dynal isolation method, from the peripheral blood of patients with metastatic breast cancer compared to similar isolates from normal blood samples. Copies of mammaglobin per ng P-actin are shown for thirty three metastatic and 11 normal samples, as indicated.

FIGS. 18A and 18B are a table showing the results of experiments in which T cell proliferation was measured. The T cells of indicated cell lines were primed with a priming peptide as indicated, then incubated with one of the following: 1A-7A (a pool of seven peptides); 3A (peptide 3A); 5A (peptide 5A); 7A (peptide 7A); mgb B 5A (a peptide corresponding to 5A, but from the same region of the mammaglobin B sequence); Hmamm10; Hmamm1; Hmamm6.

FIG. 19 shows three bar graphs in which cell lines 13, 14 and 27 were stimulated with one of the following: medium, recombinant mammoglobin, native mammoglobin, deglycosylated native mammoglobin, MB415 lysate, and SKOV3 lysate. SI=Stimulation Index.

FIG. 20 is a schematic diagram of recombinant full-length mammoglobin.

FIG. 21 shows the results of SDS-PAGE analysis of recombinant full-length mammoglobin expression, stained with Coomassie blue.

### DETAILED DESCRIPTION OF THE INVENTION

As noted above, the present invention is generally directed to compositions and methods for the therapy, diagnosis and monitoring of cancer, such as breast cancer. The compositions described herein may include mammaglobin polynucleotides, polypeptides, epitopes or antibodies that specifically recognize such epitopes. The present invention is based, in part, on the discovery of certain specific epitopes of human mammaglobin, and antibodies that bind such epitopes. The invention is further based, in part, on the discovery of antibodies that bind mammaglobin in a glycosylation-sensitive manner. Other methods described herein employ techniques for detecting mammaglobin nucleic acid in patient blood, or fractions thereof. These discoveries, within the context of the present invention, permit the generation of antibodies suited for diagnostic purposes, improved therapies for breast cancer, as well as diagnostic methods that can be based on the detection of mammaglobin RNA in blood permits sensitive diagnosis of breast cancer.

Mammaglobin Polynucleotides

The diagnostic methods provided herein generally employ mammaglobin polynucleotides (e.g., oligonucleotides) as probes or primers to detect the level of mammaglobin nucleic acid in a sample obtained from a patient. A mammaglobin oligonucleotide may encode a portion of a mammaglobin protein (e.g., at least 15, 30 or 45 consecutive nucleotides). Oligonucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., a portion of endogenous mammaglobin) or may comprise a variant of such a sequence. Polynucleotide variants may contain one or more substitutions, additions, deletions and/or insertions such that the ability of the polynucleotide to hybridize to a mammaglobin polynucleotide under assay conditions is not substantially diminished. Preferably, such polynucleotide variants are capable of hybridizing under moderately stringent conditions to a naturally occurring DNA sequence encoding a native mammaglobin (or a complementary sequence). Suitable moderately stringent conditions include prewashing in a solution of 5× SSC, 0.5% SDS, 1.0 mM EDTA (pH 8.0); hybridizing at 50° C.-65° C., 5× SSC, overnight; followed by washing twice at 65° C. for 20 minutes with each of 2×, 0.5× and 0.2× SSC containing 0.1% SDS.

Polynucleotides may be prepared using any of a variety of techniques. For example, polynucleotides may be amplified from cDNA prepared from cells expressing mammaglobin, such as breast tumor cells. Such polynucleotides may be amplified via polymerase chain reaction (PCR). For this approach, sequence-specific primers may be designed based on known mammaglobin sequences, and may be purchased or synthesized.

A portion of a coding sequence or a complementary sequence may be designed as a probe or primer to detect gene expression. Probes may be labeled by a variety of reporter groups, such as radionuclides and enzymes, and are preferably at least 10 nucleotides in length, more preferably at least 20 nucleotides in length and still more preferably at least 30 nucleotides in length. Primers are preferably 22-30 nucleotides in length.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Mammaglobin Epitopes and Polypeptides

Within the context of the present invention, polypeptides comprise at least one mammaglobin epitope, or a variant thereof. An “epitope” is a portion of mammaglobin to which one or more antibodies within an anti-mammaglobin antiserum specifically binds, or with which one or more mammaglobin-specific T cells specifically reacts, as described herein. An epitope may, but need not, be specifically bound by an antibody in a glycosylation-sensitive manner (i.e., the antibody may bind to a glycosylated epitope, to a deglycosylated epitope or to both). Polypeptides comprising a mammaglobin epitope generally comprise at least 7 consecutive amino acid residues of human mammaglobin, and preferably 9-30 consecutive amino acid residues of human mammaglobin. It should be noted that the size of an epitope may vary depending on whether the epitope is recognized by CD4⁺ T cells , CD8⁺ T cells or antibodies. In general, however, a 9-amino acid sequence is sufficient for CTL recognition. Polypeptides as described herein may be of any length. Additional sequences derived from the native protein and/or heterologous sequences may be present, and such sequences may (but need not) possess further immunogenic or antigenic properties.

Certain preferred epitopes comprise one of the following sequences, or a portion thereof that comprises at least 7, preferably at least 9 and more preferably at least 15 consecutive amino acid residues of such a sequence:

IDELKECFLNQTDETLSNVE (Pro2; SEQ ID NO: 1);

TTNAIDELKECFLNQ (Pro2-3; SEQ ID NO: 2);

SQHCYAGSGCPLLENVISKTI (Pro5; SEQ ID NO: 3);

EYKELLQEFIDDNATTNAID (peptide 5A; SEQ ID NO: 4) or

KLLMVLMLA (mgb 1; SEQ ID NO: 5).

TLSNVECFM (SEQ ID NO:50)

Other preferred epitopes comprise a glycosylation site of mammaglobin. Such epitopes are particularly useful for the generation of antibodies that specifically bind to glycosylated mammaglobin. Two such sites are the N-linked glycosylation sites asparagine (Asp)-53 (QEFIDDNATTNAI) (SEQ ID NO: 6) and Asp-68 (LKECFLNQTDETL) (SEQ ID NO: 7). Other such sites may be readily identified using, for example, an antibody library comprising antibodies to different glycosylation combinations. The binding of such antibodies to native mammaglobin from breast carcinoma cell lines may be assayed using conventional ELISA and blotting techniques. Established biochemical techniques may also be used to identify other mammaglobin glycosylation sites.

As noted above, a polypeptide may comprise a variant of a native mammaglobin epitope. A “variant,” as used herein, differs from a native epitope in one or more substitutions, deletions, additions and/or insertions, such that the ability of the variant to be bound by an antibody specific for the epitope is not substantially diminished. In other words, the ability of a variant to react with epitope-specific antisera or isolated antibodies may be enhanced or unchanged, relative to the native protein, or may be diminished by less than 50%, and preferably less than 20%, relative to the native protein. Such variants may generally be identified by modifying an epitope as provided herein and evaluating the reactivity of the modified epitope with epitope-specific antibodies or antisera as described herein. Preferred variants include those in which substitutions are made at no more than 20% of the residues in the epitope.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptides may be prepared using any of a variety of well known techniques. Recombinant polypeptides may be readily prepared from mammaglobin DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast or a mammalian cell line such as COS or CHO. Supernatants from suitable host/vector systems that secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Polypeptides having fewer than about 100 amino acids, and generally fewer than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Applied BioSystems, Inc. (Foster City, Calif.), and may be operated according to the manufacturer's instructions.

Within related aspects, polynucleotides that encode a polypeptide as provided herein are provided. In general, polypeptides and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application No. 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application No. 60/158,585; see also, Skeiky et al., Infection and Immun. (1999) 67:3998-4007, incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NSI (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

Antibodies and Fragments Thereof

The present invention further provides agents, such as antibodies and antigen-binding fragments thereof, that specifically bind to a mammaglobin epitope. As used herein, an antibody, or antigen-binding fragment thereof, is said to “specifically bind” to a mammaglobin epitope if it reacts at a detectable level (within, for example, an ELISA) with the epitope, and does not react detectably with unrelated proteins under similar conditions. Preferred antibodies bind detectably to an epitope of mammaglobin, but do not bind detectably to other portions of mammaglobin that do not overlap with the epitope (or that overlap by less than five amino acid residues). As used herein, “binding” refers to a noncovalent association between two separate molecules such that a complex is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 10³ L/mol. The binding constant maybe determined using methods well known in the art.

Binding agents may be further capable of differentiating between patients with and without a cancer, such as breast cancer, using the representative assays provided herein. In other words, antibodies or other binding agents that bind to a mammaglobin epitope will generate a signal indicating the presence of a cancer in at least about 20% of patients with the disease, and will generate a negative signal indicating the absence of the disease in at least about 90% of individuals without the cancer. To determine whether a binding agent satisfies this requirement, biological samples (e.g., blood, sera, urine and/or tumor biopsies) from patients with and without a cancer (as determined using standard clinical tests) may be assayed as described herein for the presence of polypeptides that bind to the binding agent. It will be apparent that a statistically significant number of samples with and without the disease should be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, and RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Certain preferred monoclonal antibodies specifically bind to an epitope sequence recited above (Pro2, Pro2-3, Pro5, peptide 5A or mgb 1). Such antibodies include the rabbit antibodies designated 29C11, 6A1, 2D3 and 16D8 and the mouse antibody designated 197-1H11 herein. Other preferred antibodies bind to other sequences, such as conformationally dependent sequences. Such antibodies include those designated 31-1H7, 32-IG11, 304-1A5 and 98-1F4 herein. Other preferred antibodies bind to a glycosylation site of mammaglobin with an affinity that is dependent on glycosylation. For example, certain antibodies specifically bind to glycosylated mammaglobin (i.e., require glycosylation of a particular glycosylation site for optimal binding). As used herein, an antibody, or antigen binding fragment thereof, specifically binds to glycosylated mammaglobin if it binds to a glycosylated mammaglobin with an affinity that is at least two-fold, preferably at least five-fold, greater than the affinity with which it binds deglycosylated mammaglobin (mammaglobin that is enzymatically deglycosylated, using well known techniques, so as to remove substantially all glycosylation). Glycosylation results when oligosaccharide units are attached to the protein via asparagine (N-linked) or serine and threonine residues (O-linked). Compared to normal cells, protein glycosylation is often altered in tumor cells. This difference in protein glycosylation can be exploited to provide a tumor-specific antibody for diagnostic purposes (e.g., for the diagnosis of breast cancer). This is particularly true for heavily glycosylated proteins, such as mammaglobin. Although the predicted molecular weight of mammaglobin is 9.2 kDa, the mature form of this protein expressed in breast carcinoma cells runs at a molecular weight of approximately 18-25 kDa. It has been found, within the context of the present invention, that the additional molecular weight of mammaglobin is due to the attachment of oligosaccharides. Thus, roughly one half or more of the molecular weight of mammaglobin is due to glycosylation.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988) and digested by papain to yield Fab and Fc fragments. The Fab and Fc fragments may be separated by affinity chromatography on protein A bead columns.

Monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

A variety of routes of administration for the antibodies and immunoconjugates may be used. Typically, administration will be intravenous, intramuscular, subcutaneous or in the bed of a resected tumor. It will be evident that the precise dose of the antibody/immunoconjugate will vary depending upon the antibody used, the antigen density on the tumor, and the rate of clearance of the antibody.

T Cells

Immunotherapeutic compositions may also, or alternatively, comprise T cells that recognize mammaglobin. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures. Briefly, T cells, which may be isolated from a patient or a related or unrelated donor by routine techniques (such as by Ficoll/Hypaque density gradient centrifugation of peripheral blood lymphocytes), are incubated with a mammaglobin polypeptide. For example, T cells may be incubated in vitro for 2-9 days (typically 4 days) at 37° C. with a mammaglobin polypeptide (e.g., 5 to 25 μg/ml) or cells synthesizing a comparable amount of mammaglobin polypeptide. It may be desirable to incubate a separate aliquot of a T cell sample in the absence of mammaglobin polypeptide to serve as a control.

T cells may be stimulated with a mammaglobin polypeptide, polynucleotide encoding a mammaglobin polypeptide and/or an antigen presenting cell (APC) that expresses such a polypeptide. Such stimulation is performed under conditions and for a time sufficient to permit the generation of T cells that are specific for the polypeptide. Preferably, a mammaglobin polypeptide or polynucleotide is present within a delivery vehicle, such as a microsphere, to facilitate the generation of specific T cells.

T cells are considered to be specific for a mammaglobin polypeptide if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Other ways to detect T cell proliferation include measuring increases in interleukin-2 (IL-2) production, Ca²⁺ flux, or dye uptake, such as 3-(4,5-dimethylthiazol-2-yl)-2,5-diphenyl-tetrazolium. Alternatively, synthesis of lymphokines (such as interferon-gamma) can be measured or the relative number of T cells that can respond to a mammaglobin polypeptide may be quantified. Contact with a mammaglobin polypeptide (100 ng/ml-100 μg/ml, preferably 200 ng/ml-25 μg/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). Mammaglobin-specific T cells may be expanded using standard techniques. Within preferred embodiments, the T cells are derived from a patient, a related donor or an unrelated donor, and are administered to the patient following stimulation and expansion.

T cells that have been activated in response to a mammaglobin polypeptide, polynucleotide or polypeptide-expressing APC may be CD4⁺ and/or CD8⁺. Specific activation of CD4⁺ or CD8⁺ T cells may be detected in a variety of ways. Methods for detecting specific T cell activation include detecting the proliferation of T cells, the production of cytokines (e.g., lymphokines), or the generation of cytolytic activity (i.e., generation of cytotoxic T cells specific for mammaglobin). For CD4⁺ T cells, a preferred method for detecting specific T cell activation is the detection of the proliferation of T cells. For CD8⁺ T cells, a preferred method for detecting specific T cell activation is the detection of the generation of cytolytic activity.

For therapeutic purposes, CD4⁺ or CD8⁺ T cells that proliferate in response to a mammaglobin polypeptide, polynucleotide or APC can be expanded in number either in vitro or in vivo. Proliferation of such T cells in vitro may be accomplished in a variety of ways. For example, the T cells can be re-exposed to a mammaglobin polypeptide (e.g., a short peptide corresponding to an immunogenic portion of such a polypeptide) with or without the addition of T cell growth factors, such as interleukin-2, and/or stimulator cells that synthesize a mammaglobin polypeptide. The addition of stimulator cells is preferred where generating CD8⁺ T cell responses. T cells can be grown to large numbers in vitro with retention of specificity in response to intermittent restimulation with mammaglobin polypeptide. Briefly, for in vitro stimulation, lymphocytes may be placed in a vessel with media containing human serum, mammoglobin protein or peptide and cytokines such as IL-2, IL-10 and IL-7. Cells may be incubated for seven to fourteen days and then restimulated in a similar manner using autologous antigen presenting cells, mammoglobin protein or peptide and cytokines. Antigen specific T cells may also be expanded in vitro using either antigen or a mitogen or non-specific stimulator such as α-CD3 or PHA.

Alternatively, one or more T cells that proliferate in the presence of mammaglobin polypeptide can be expanded in number by cloning. Methods for cloning cells are well known in the art, and include limiting dilution. Responder T cells may be purified from the peripheral blood of sensitized patients by density gradient centrifugation and sheep red cell rosetting and established in culture by stimulating with the nominal antigen in the presence of irradiated autologous filler cells. In order to generate CD4⁺ T cell lines, mammaglobin polypeptide is used as the antigenic stimulus and APC derived from autologous peripheral blood lymphocytes (PBL) or lymphoblastoid cell lines (LCL) immortalized by infection with Epstein Barr virus are used as antigen presenting cells. In order to generate CD8⁺ T cell lines, autologous antigen-presenting cells transfected with an expression vector that produces mammaglobin polypeptide may be used as stimulator cells. Established T cell lines may be cloned following antigen stimulation by plating stimulated T cells at a frequency of 0.5 cells per well in 96-well flat-bottom plates with 1×10⁶ irradiated PBL or LCL cells and recombinant interleukin-2 (rIL2) (50 U/ml). Wells with established clonal growth may be identified at approximately 2-3 weeks after initial plating and restimulated with appropriate antigen in the presence of autologous antigen-presenting cells, then subsequently expanded by the addition of low doses of rIL2 (10 U/ml) 2-3 days following antigen stimulation. T cell clones may be maintained in 24-well plates by periodic restimulation with antigen and rIL2 approximately every two weeks. Cloned and/or expanded cells may be administered back to the patient as described, for example, by Chang et al., Crit. Rev. Oncol. Hematol. 22:213, 1996.

Within certain embodiments, allogeneic T-cells may be primed (i.e., sensitized to mammaglobin) in vivo and/or in vitro. Such priming may be achieved by contacting T cells with a mammaglobin polypeptide, a polynucleotide encoding such a polypeptide or a cell producing such a polypeptide under conditions and for a time sufficient to permit the priming of T cells. In general, T cells are considered to be primed if, for example, contact with a mammaglobin polypeptide results in proliferation and/or activation of the T cells, as measured by standard proliferation, chromium release and/or cytokine release assays as described herein. A stimulation index of more than two fold increase in proliferation or lysis, and more than three fold increase in the level of cytokine, compared to negative controls, indicates T-cell specificity. Cells primed in vitro may be employed, for example, within a bone marrow transplantation or as donor lymphocyte infusion.

Pharmaceutical Compositions and Vaccines

Within certain aspects, polypeptides, polynucleotides, T cells and/or binding agents described herein may be incorporated into pharmaceutical compositions or immunogenic compositions (i.e., vaccines). Alternatively, a pharmaceutical composition may comprise an antigen-presenting cell (e.g., a dendritic cell) transfected with a mammaglobin polynucleotide such that the antigen presenting cell expresses a mammaglobin polypeptide. Pharmaceutical compositions comprise one or more such compounds and a physiologically acceptable carrier. Vaccines may comprise one or more such compounds and an immunostimulant. An immunostimulant may be any substance that enhances or potentiates an immune response (antibody- and/or cell-mediated) to an exogenous antigen. Examples of immunostimulants include adjuvants, biodegradable microspheres (e.g., polylactic galactide) and liposomes (into which the compound is incorporated; see e.g., Fullerton, U.S. Pat. No. 4,235,877). Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive. For example, one or more immunogenic portions of other tumor antigens may be present, either incorporated into a fusion polypeptide or as a separate compound, within the composition or vaccine.

A pharmaceutical composition or vaccine may contain DNA encoding one or more of the polypeptides as described above, such that the polypeptide is generated in situ. As noted above, the DNA may be present within any of a variety of delivery systems known to those of ordinary skill in the art, including nucleic acid expression systems, bacteria and viral expression systems and mammalian expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells. It will be apparent that a vaccine may comprise both a polynucleotide and a polypeptide component. Such vaccines may provide for an enhanced immune response.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. Compounds may also be encapsulated within liposomes using well known technology.

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555 and WO 99/33488. Immunostimulatory DNA sequences are also described, for example, by Sato et al., Science 273:352, 1996. Another preferred adjuvant is a saponin, preferably QS21 (Aquila Biopharmaceuticals Inc., Frainingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa Corporation; Seattle, Wash.), RC-529 (Corixa Corporation; Seattle, Wash.) and aminoalkyl glucosaminide 4-phosphates (AGPs).

Any vaccine provided herein may be prepared using well known methods that result in a combination of antigen, immunostimulant and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 14:1429-1438, 1996) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding a mammaglobin protein (or portion or other variant thereof) such that the mammaglobin polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the mammaglobin polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

Cancer Therapy

In further aspects of the present invention, the compositions described herein may be used for immunotherapy of cancer, such as breast cancer. Within such methods, pharmaceutical compositions and vaccines are typically administered to a patient. As used herein, a “patient” refers to any warm-blooded animal, preferably a human. A patient may or may not be afflicted with cancer. Accordingly, the above pharmaceutical compositions and vaccines may be used to prevent the development of a cancer or to treat a patient afflicted with a cancer. A cancer may be diagnosed using criteria generally accepted in the art, including the presence of a malignant tumor. Pharmaceutical compositions and vaccines may be administered either prior to or following surgical removal of primary tumors and/or treatment such as administration of radiotherapy or conventional chemotherapeutic drugs.

Within certain embodiments, immunotherapy may be active immunotherapy, in which treatment relies on the in vivo stimulation of the endogenous host immune system to react against tumors with the administration of immune response-modifying agents (such as tumor vaccines, bacterial adjuvants and/or cytokines).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

The polypeptides provided herein may also be used to generate and/or isolate tumor-reactive T cells, which can then be administered to a patient. In one such technique, antigen-specific T cell lines may be generated by in vivo immunization with short peptides corresponding to immunogenic portions of the disclosed polypeptides. The resulting antigen-specific CD8⁺ CTL clones may be isolated from the patient, expanded using standard tissue culture techniques and returned to the patient.

Within another embodiment, syngeneic or autologous dendritic cells may be pulsed with peptides corresponding to at least an immunogenic portion of a polypeptide disclosed herein. The resulting antigen-specific dendritic cells may either be transferred into a patient or employed to stimulate T cells to provide antigen-specific T cells which may, in turn, be administered to a patient. The use of peptide-pulsed dendritic cells to generate antigen-specific T cells and the subsequent use of such antigen-specific T cells to eradicate tumors in a murine model has been demonstrated by Cheever et al., Immunological Reviews 157:177, 1997.

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 100 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to mammaglobin generally correlate with an improved clinical outcome. Such immune responses may be evaluated using standard proliferation, cytotoxicity or cytokine assays, which may be performed using samples obtained from a patient before and after treatment.

Methods for Detecting Cancer

In general, a cancer may be detected in a patient based on the presence of one or more mammaglobin epitopes or antibodies thereto in a biological sample obtained from the patient. In other words, such epitopes may be used as markers to indicate the presence or absence of a cancer such as breast cancer. In general, such an epitope or antibody should be present at a level that is at least three fold higher in tumor tissue than in normal tissue.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent.

The solid support may be any material known to those of ordinary skill in the art to which the binding agent may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay or a capture antibody (ELISA) assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with breast cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as breast cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

For certain embodiments (e.g., sandwich assays), quantitative measurements of antigen may be obtained. Within such embodiments, a standard curve may be generated. Signals obtained for antigen levels in particular samples may then be compared to the standard curve, to allow quantitation. The cut-off value within such assays may be an amount of mammaglobin indicative of the presence of breast cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the epitopes and binding agents of the present invention. The above descriptions are intended to be exemplary only. For example, it will be apparent to those of ordinary skill in the art that the above protocols may be readily modified to use polypeptides as described herein to detect antibodies that bind to such polypeptides in a biological sample. The detection of such mammaglobin epitope-specific antibodies may correlate with the presence of a cancer. Other preferred assay protocols include laser scanning cytometry (a microscopic technique in which cells are stained with labeled antibody) and immunohistochemical detection. Such techniques may generally be performed according to techniques known in the art. Antibodies as provided herein may further be used to facilitate cell identification and sorting in vitro, permitting the selection of cells expressing mammaglobin (or varying levels of mammaglobin). Preferably, antibodies for use in such methods are linked to a detectable marker. Suitable markers are well known in the art and include radionuclides, luminescent groups, fluorescent groups, enzymes, dyes, constant immunoglobulin domains and biotin. Within one preferred embodiment, an antibody linked to a fluorescent marker, such as fluorescein, is contacted with the cells, which are then analyzed by fluorescence activated cell sorting (FACS).

In another embodiment, the above polypeptides may be used as markers for the progression of cancer. In this embodiment, assays as described above for the diagnosis of a cancer may be performed over time, and the change in the level of reactive polypeptide(s) evaluated. For example, the assays may be performed every 24-72 hours for a period of 6 months to 1 year, and thereafter performed as needed. In general, a cancer is progressing in those patients in whom the level of polypeptide detected by the binding agent increases over time. In contrast, the cancer is not progressing when the level of reactive polypeptide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications.

To improve sensitivity, assays as described herein may be combined with assays to detect other tumor-associated antigens. It will be apparent that binding agents specific for different proteins may be combined within a single assay. The selection of tumor protein markers may be based on routine experiments to determine combinations that results in optimal sensitivity.

By alternative embodiments of the present invention, a cancer may be detected in a patient based on the presence of mammaglobin polynucleotides in a biological sample obtained from the patient. In other words, such polynucleotides may be used as markers to indicate the presence or absence of a cancer such as breast cancer. In particular, polynucleotide primers and probes may be used to detect the level of mRNA encoding mammaglobin, which is indicative of the presence or absence of breast cancer. In general, the presence of a mammaglobin polynucleotide at a level that is at least two fold, preferably at least three fold, higher than in normal tissue is indicative of breast cancer.

There are a variety of biological samples that may be used for an assay provided herein, including various body fluids and tumor samples. Preferred samples are blood, and fractions thereof, such as peripheral blood, serum or plasma. In general, RNA may be isolated from blood or a fraction thereof using any standard technique.

Prior to PCR or hybridization analysis, a sample is treated by any standard technique to remove epithelial cells. It has been found, within the context of the present invention, that such treatment improves the sensitivity of the assay by up to 10 fold. One method for removing epithelial cells employs Dynal's Epithelial cell enrichment beads (Dynal, Oslo, Norway), which may be used according to the manufacturer's instructions. Preferred samples for analysis are patient whole blood samples, from which epithelial cells have been removed.

Within certain embodiments, at least two oligonucleotide primers may be employed in a polymerase chain reaction (PCR) based assay to amplify a portion of a mammaglobin cDNA derived from a biological sample, wherein at least one of the oligonucleotide primers is specific for (i.e., hybridizes to) a polynucleotide encoding mammaglobin. The amplified cDNA is then separated and detected using techniques well known in the art, such as gel electrophoresis and autoradiography. Similarly, oligonucleotide probes that specifically hybridize to a mammaglobin polynucleotide may be used in a hybridization assay to detect mammaglobin expression in a biological sample.

To permit hybridization under assay conditions, oligonucleotide primers and probes should comprise an oligonucleotide sequence that has at least about 60%, preferably at least about 75% and more preferably at least about 90%, identity to a portion of a mammaglobin polynucleotide that is at least 10 nucleotides, and preferably at least 20 nucleotides, in length. Oligonucleotide primers and/or probes which may be usefully employed in the diagnostic methods described herein preferably are at least 10-40 nucleotides in length. Techniques for both PCR based assays and hybridization assays are well known in the art (see, for example, Mullis et al., Cold Spring Harbor Symp. Quant. Biol. 51:263, 1987; Erlich ed., PCR Technology, Stockton Press, NY, 1989).

One preferred assay employs RT-PCR, in which PCR is applied in conjunction with reverse transcription. Typically, RNA is extracted from a sample tissue and is reverse transcribed to produce cDNA molecules. PCR amplification using at least one specific primer generates a cDNA molecule, which may be separated and visualized using, for example, gel electrophoresis. Amplification may be performed on samples obtained from biological samples taken from a test patient and an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

Yet another amplification technique that may be used within such assays is real-time PCR (see Gibson et al., Genome Research 6:995-1001, 1996; Heid et al., Genome Research 6:986-994, 1996). Real-time PCR is a technique that evaluates the level of PCR product accumulation during amplification, permitting quantitative evaluation of mRNA levels. Briefly, mRNA is initially extracted from cells of interest using standard techniques. Real-time PCR may then be performed, for example, using a Perkin Elmer/Applied Biosystems (Foster City, Calif.) 7700 Prism instrument. Matching primers and fluorescent probes may be designed for mammaglobin using, for example, the primer express program provided by Perkin Elmer/Applied Biosystems (Foster City, Calif.). Optimal concentrations of primers and probes may be initially determined by those of ordinary skill in the art, and control (e.g., β-actin) primers and probes may be obtained commercially from, for example, Perkin Elmer/Applied Biosystems (Foster City, Calif.). To quantitate the amount of mammaglobin RNA in a sample, a standard curve may be generated alongside using a plasmid containing a mammaglobin gene. Standard dilutions ranging from 10-10⁶ copies of the gene of interest are generally sufficient. In addition, a standard curve may be generated for the control sequence, to permit standardization of initial RNA content of a tissue sample to the amount of control for comparison purposes.

Certain in vivo diagnostic assays may be performed directly on a tumor. One such assay involves contacting tumor cells with a polynucleotide probe. Bound probe may be detected directly or indirectly using a reporter group.

As noted above, to improve sensitivity, multiple breast tumor protein markers may be assayed within a given sample. For example, a polynucleotide probe or primer as described herein may bc used concurrently with a probe or primer designed to detect a different marker. The selection of breast tumor markers may be based on routine experiments to determine combinations that results in optimal sensitivity.

Diagnostic Kits

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a mammaglobin epitope. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Preferred kits are those designed for use within sandwich assays. Such kits comprise two or more components for use within such assays. For example, such a kit may comprise standards based on recombinant mammaglobin for use in preparing a standard curve. Such a kit may comprise one or both antibodies for use within the assay (i.e., the capture antibody and/or signal antibody), with or without additional reagents for use in detecting mammaglobin binding.

Kits designed to detect the level of mRNA encoding mammaglobin in a biological sample may comprise at least one oligonucleotide probe or primer, as described above. Such an oligonucleotide may be used, for example, within a PCR or hybridization assay. Additional components that may be present within such kits include a second oligonucleotide and/or a diagnostic reagent or container to facilitate the detection of a mammaglobin polynucleotide.

The following Examples are offered by way of illustration and not by way of limitation.

### EXAMPLES

### Example 1

### Identification of Mammaglobin Epitopes and Preparation of Antibodies

This Example illustrates the preparation of anti-mammaglobin antibodies and epitope mapping.

Rabbits and mice were immunized with full-length human mammaglobin protein. Mouse monoclonal antibodies were isolated with standard hybridoma technology. Rabbit monoclonal antibodies were isolated with selected lymphocyte antibody method (SLAM) technology. In addition to these antibodies, a purified polyclonal antibody directed against the C-terminus of mammaglobin was also developed following immunization of rabbits with a C-terminal peptide.

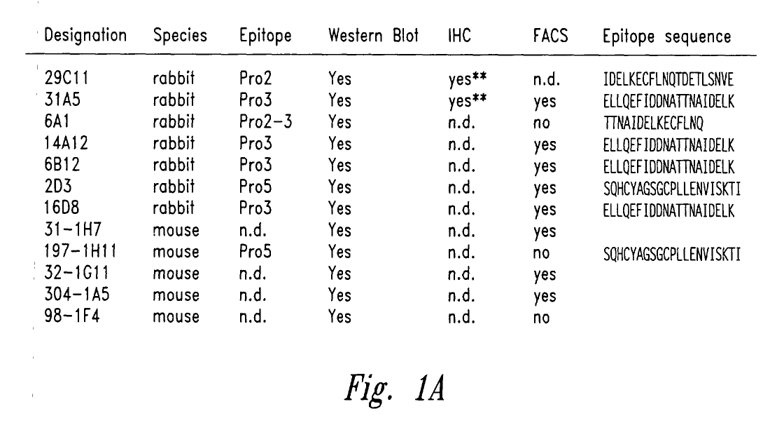

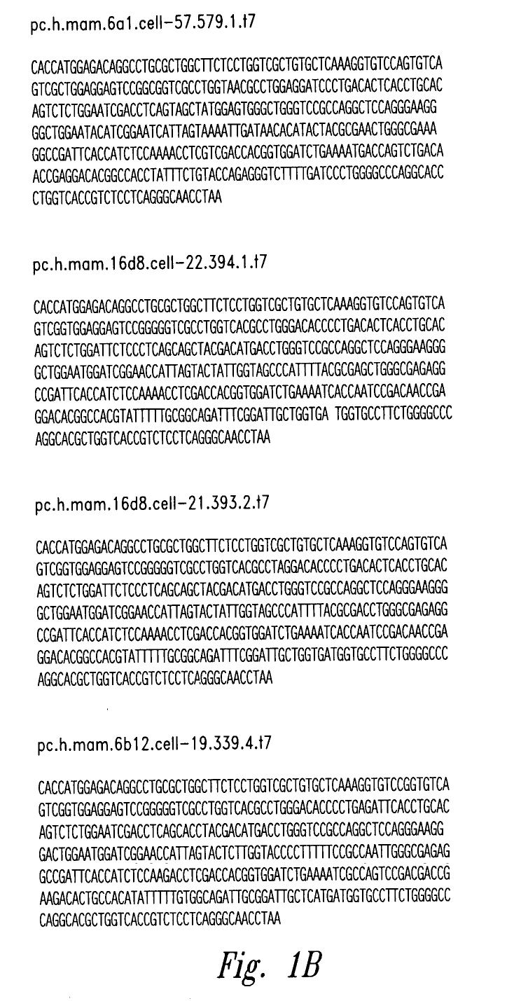

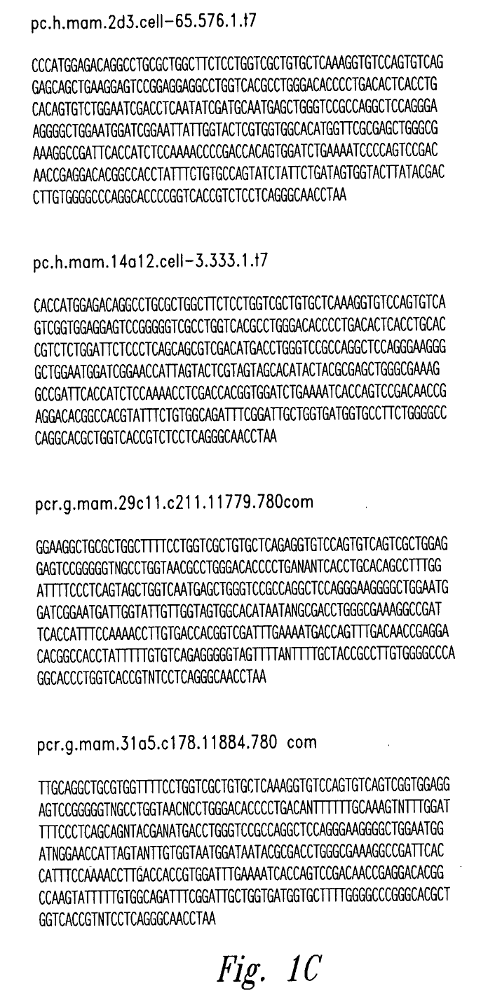

FIG. 1A illustrates the monoclonal antibodies that were developed for mammaglobin. For the rabbit monoclonal antibodies the Ig variable regions were sequenced. The sequence for the variable regions of each rabbit antimammaglobin monoclonal antibody is shown in FIGS. 1B-1C.

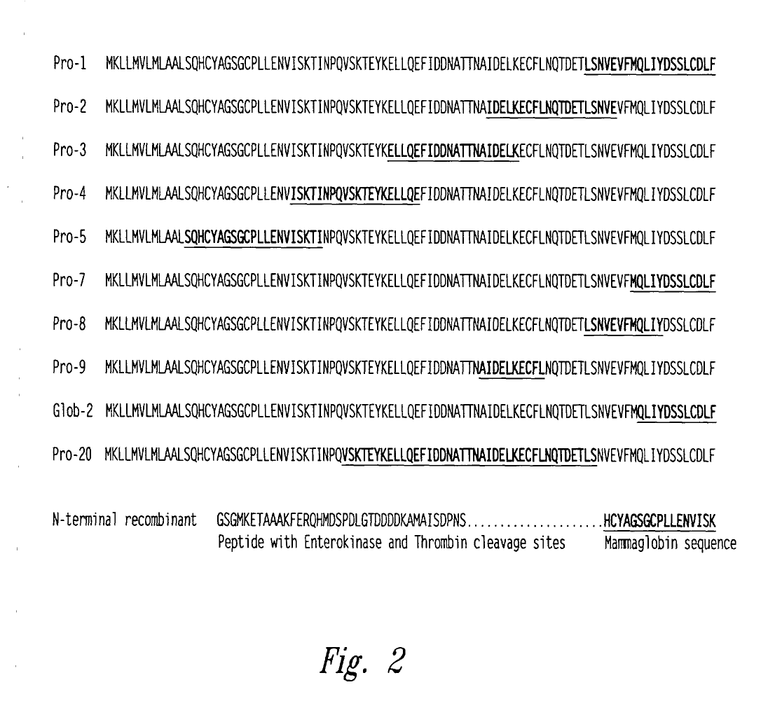

In order to better define the epitope binding region of each monoclonal antibody a series of peptides was generated that spans the entire mammaglobin protein sequence. The amino acid sequence for mammaglobin is shown in FIG. 2, and the corresponding peptides are indicated. In addition to the peptides, a short recombinant form of mammaglobin was generated by cleavage with protease. 96 well microtiter plates (Costar) were coated with either peptide or recombinant antigen at 200 ng/well. Coating was overnight at 4° C. Plates were then aspirated and blocked with phosphate buffered saline containing 1% (w/v) BSA for 2 hours at room temperature, and then washed in PBS containing 0.1% Tween 20 (PBST). Purified rabbit antibodies at different dilutions (1000 to 7.8 ng/ml) in PBST was added to the wells and incubated for 30 minutes at room temperature. This was followed by washing 6 times with PBST and then incubating with Protein-A HRP conjugated at a 1/20000 dilution for a further 30 minutes. Plates were washed 6 times in PBST and then incubated with Tetramethylbenzidine (TMB) substrate for a further 15 minutes. The reaction was stopped by the addition of 1 N sulfuric acid and plates were read at 450 nm using an ELISA plate reader.

ELISA with the mouse monoclonal antibodies was performed with supernatants from tissue culture run neat in the assay.

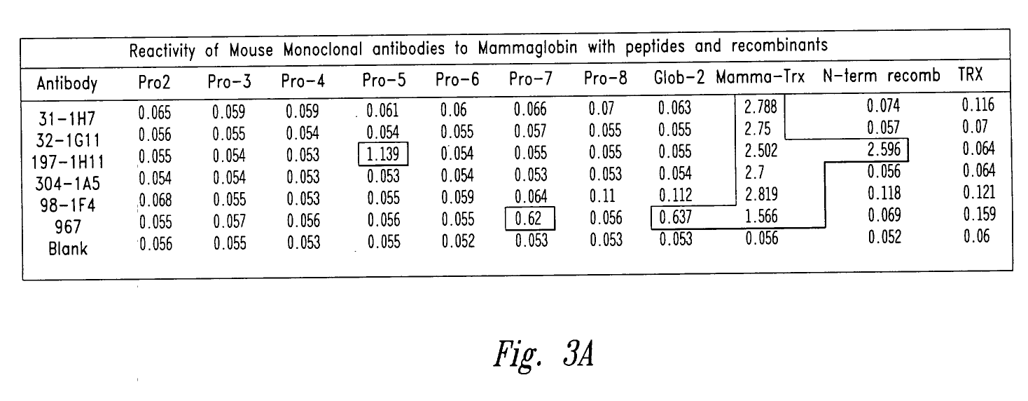

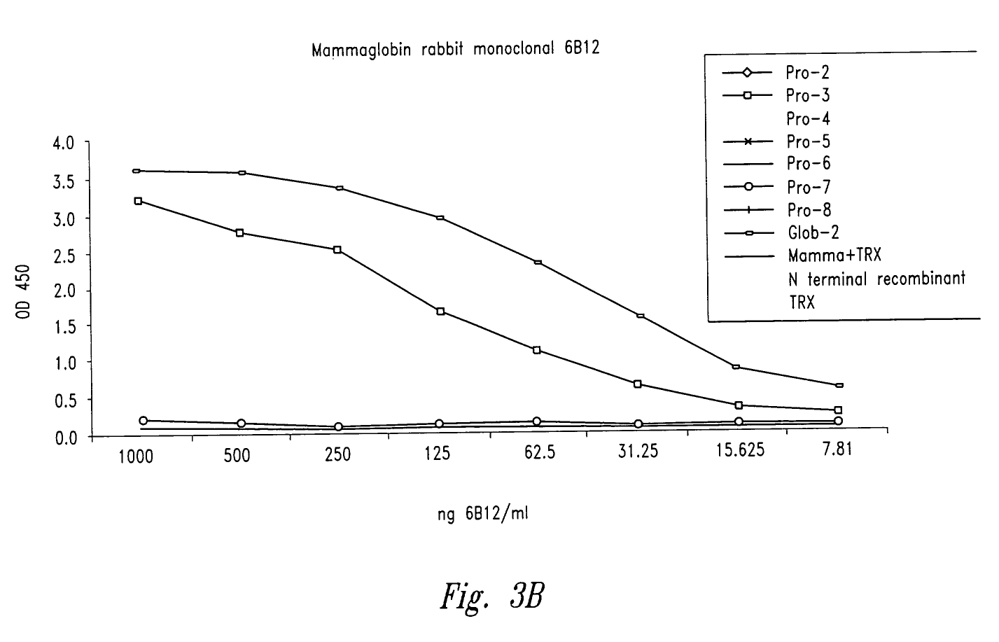

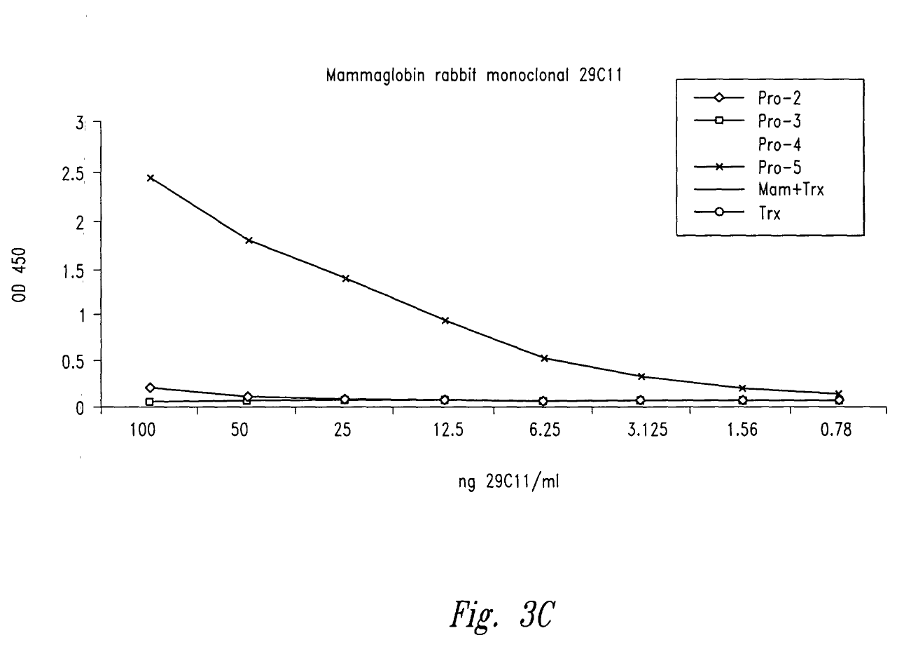

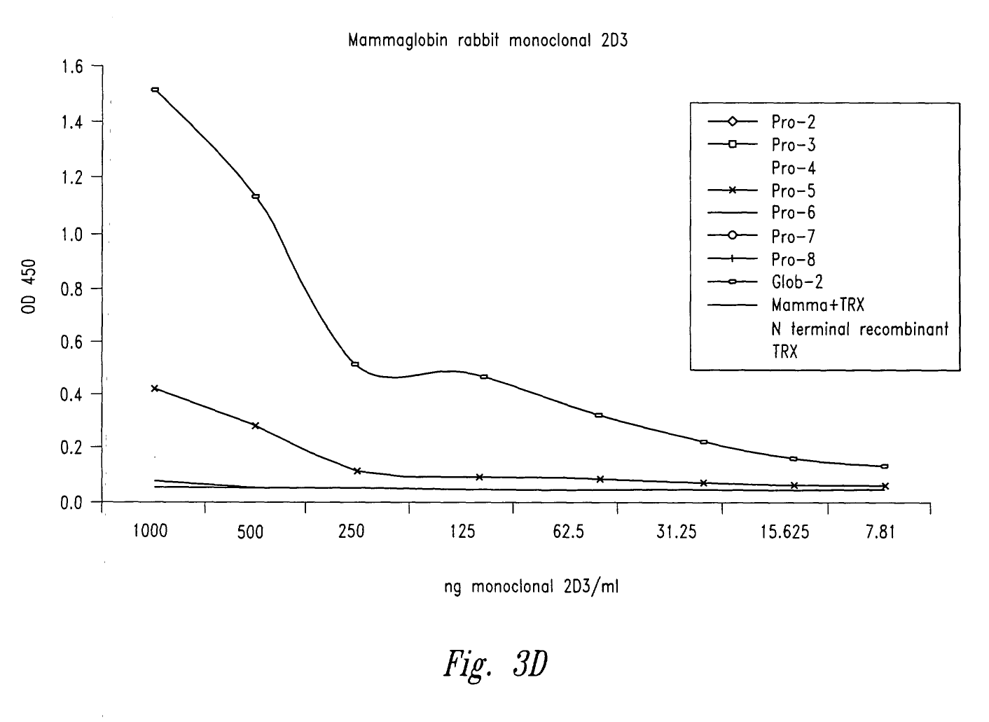

A summary of the data is shown in FIG. 3A. Shaded cells are considered positive for the antibody. The reactivity of three different epitopes is shown in FIGS. 3B, 3C and 3D, where 2D3 reacts with pro5 and the N-terminal recombinant and 29C11 reacts weakly with pro2. The epitope binding sites of the antimammaglobin antibodies are summarized in FIG. 1A.

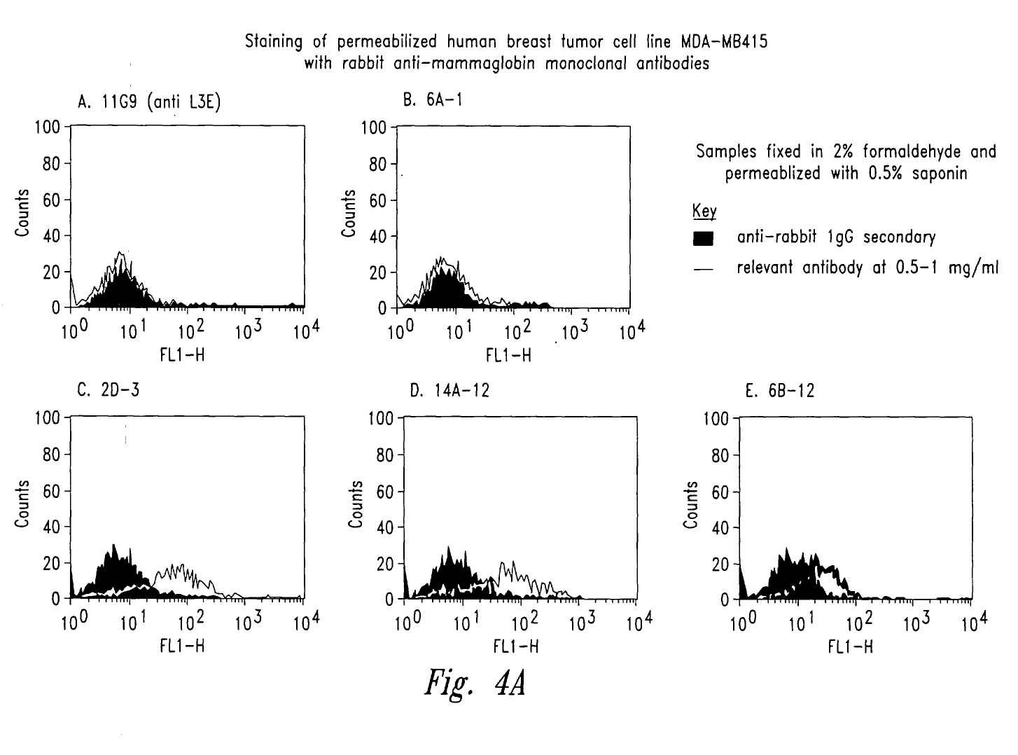

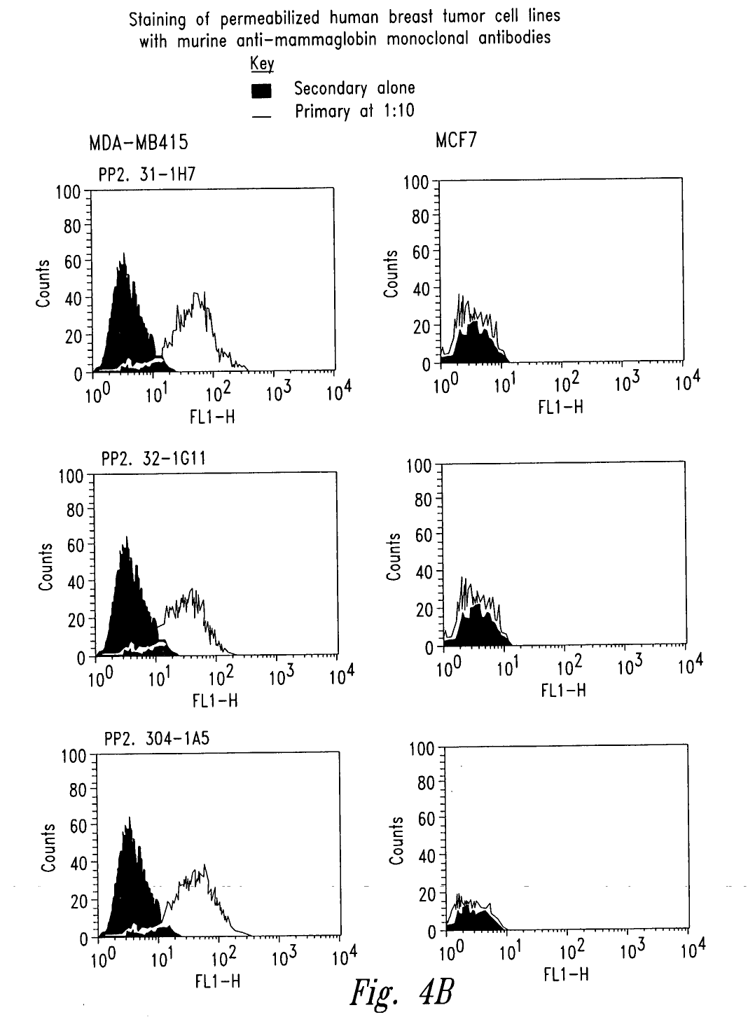

Subsequent to epitope mapping, the antibodies were tested by FACS analysis on a cell line that expresses mammaglobin, MB415 breast carcinoma cells. In order to ensure specificity of antibody binding, MCF-7 cells that do not express mammaglobin were also tested by FACS analysis under identical conditions. Cells were fixed with 4% formaldehyde for 20 min before being washed 2 times. Cells were then permeabilized for 10 minutes with PBS containing 0-0.1% saponin. 0.5 μg of anti-mammaglobin monoclonal antibody was added and cells were incubated at room temperature for 30 minutes before being washed 2 times and incubated with a FITC-labeled goat anti-rabbit or mouse secondary antibody for 20 minutes. After being washed 2 times, cells were analyzed with an Excalibur fluorescent activated cell sorter. The results are illustrated in FIG. 4A.

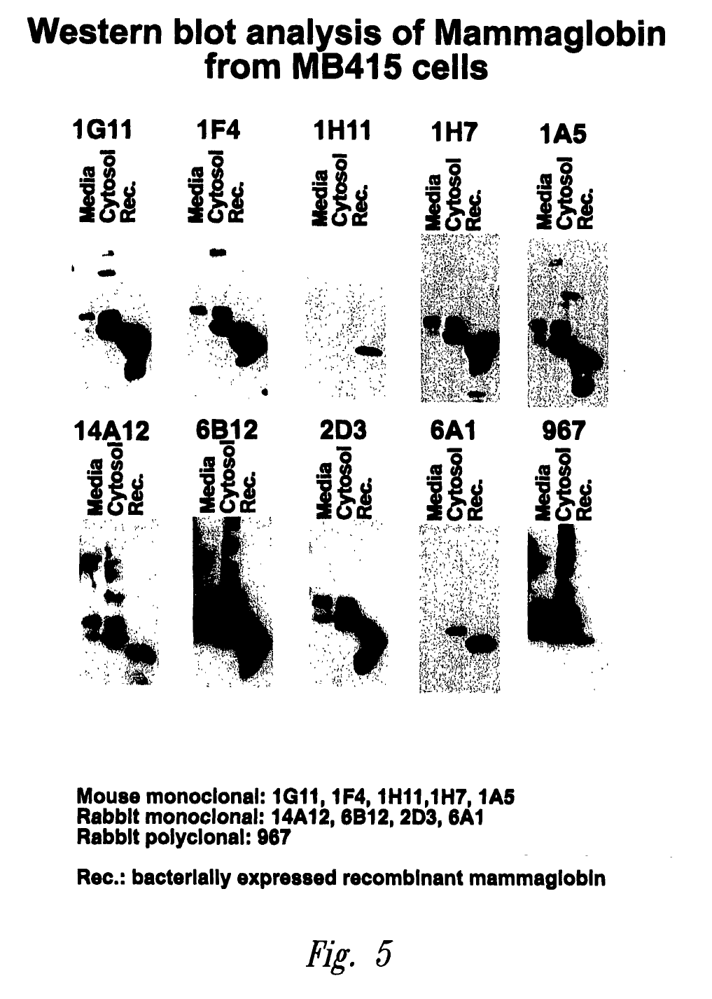

Western blot analysis was also used to characterize anti-mammaglobin monoclonal specificity (FIG. 5). SDS-PAGE was performed on 1) media in which MDA-MB-415 cells were grown, 2) MDA-MB-415 cell lysate and 3) bacterially expressed recombinant mammaglobin. Protein was transferred to nitrocellulose and then Western blotted for the antimammaglobin monoclonal antibodies at an antibody concentration of 1 μg/ml. Protein was detected using horse radish peroxidase (HRP) conjugated to either a goat anti-mouse monoclonal antibody or to protein A-Sepharose. The purified anti-mammaglobin polyclonal antibody recognized bacterial expressed recombinant mammaglobin, as well as mammaglobin expressed in and secreted from MDA-MB-415 breast carcinoma cells. All mouse and rabbit monoclonal antibodies recognized recombinant bacterial expressed mammaglobin. With the exception of 197-1H11, all of the mouse monoclonal antibodies recognized mammaglobin secreted into the cell media or expressed within the cytoplasm. The rabbit monoclonal antibodies 14A12, 6B12 and 2D3 recognized recombinant bacterial expressed mammaglobin, as well as mammaglobin expressed in the cytoplasm and secreted into the media. Although unable to recognize mammaglobin secreted into the media, rabbit monoclonal antibody 6A1 was able to recognize bacterial expressed mammaglobin and mammaglobin expressed in the cytoplasm of MB415 cells. The inability of monoclonal antibodies 197-1H11 and 6A1 to associate with specific forms of mammaglobin likely reflect differential posttranslation modifications such as glycosylation and/or relative affinity of the antibody to mammaglobin.

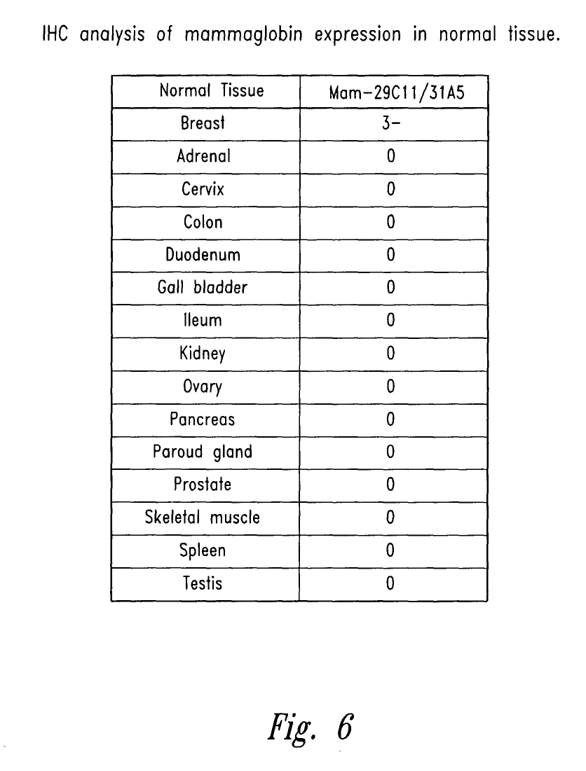

In order to determine which tissues express mammaglobin, immunohistochemistry (IHC) analysis was performed on a diverse range of tissue sections. Tissue samples were fixed in formalin solution for 24 hours and embedded in paraffin before being sliced into 10 micron sections. Tissue sections were permeabilized and incubated with anti-mammaglobin antibody for 1 hour. HRP-labeled anti-mouse or anti-rabbit antibody was used to visualize mammaglobin immunoreactivity. FIG. 6 summarizes the tissue-specific distribution of mammaglobin protein. Mammaglobin was highly expressed in breast tissue but not found in other tissues tested including adrenal, cervix, colon, duodenum, gall bladder, ileum, kidney, ovary, pancreas, parotid gland, prostate, skeletal muscle, spleen, and testis.

### Example 2

### Sandwich Immunoassays for Mammaglobin

This Example illustrates the use of antibodies provided herein for detection of mammaglobin in serum.

Monoclonal antibodies and the rabbit polyclonal antibody 967 directed to the C-terminal 16 amino acid peptide of mammaglobin were evaluated in sandwich ELISAs for their ability to detect mammaglobin in lysates of MB415 cells, cell supernatants of MB415 cells and also in the serum of breast cancer patients. Antibodies were paired based on their ability to detect different epitopes. The following describes some of the sandwich combinations tested. In all assays a standard curve was constructed by spiking recombinant mammaglobin into male serum.

Mouse/Rabbit Antibody Sandwiches

Assays were designed to capture the mouse monoclonal antibody using a solid phase of goat anti-mouse IgG as part of the sandwich. 96 well plates (Costar Corning) were coated overnight at 4° C. with 200 ng/well of goat anti mouse IgG (Rockland antibodies, Rockland, Me.). Plates were washed in Phosphate buffered saline (PBS) containing 0.05% Tween 20 (PBST) then blocked with 1% BSA in phosphate buffered saline(PBS) for 2 hours. Mouse monoclonal supernatants were then added (50 ul) at 1:10 dilution in PBS and the plates incubated for a further hour at room temperature. Plates were washed six times in PBS Tween 20 (PBST) then blocked with 1% normal mouse serum and 1% normal human serum for 1 hour then washed again. Samples and standards were applied to the wells and the plate was incubated for 1 hour at room temperature. The plate was then washed six times in PBS containing 0.05% Tween. Biotinylated 31A5 or 2D3 were used as the conjugate at lug/ml in PBS and 1% Normal mouse serum. These were incubated for 1 hour at room temperature then washed six times in PBST. 50 μl of a 1:10000 dilution of streptavidin HRP in PBS containing 1% normal mouse serum was added and the plate incubated at room temperature for 30 minutes at which time the plate was again washed six times. TMB (tetramethyl benzidine) substrate (Kirkegaard and Perry) was then added to the well and incubated for a further 15 minutes. The reaction was stopped with 1N H₂SO₄ (100 μl) and the signal generated read at 450 nm. A standard curve relating pg mammaglobin in the assay was constructed using recombinant mammaglobin spiked into normal male serum and samples run in the test were quantitated using this standard curve.

Rabbit/Rabbit Antibody Sandwiches

Two assays were performed. The first utilized affinity purified rabbit anti 967 peptide (C-terminal 16 amino acid peptide) as the solid phase and 2D3 rabbit monoclonal biotinylated as the signal antibody. The second used 2D3 as the solid phase antibody and 31A5-biotinylated as the signal antibody. In the first assay the affinity purified polyclonal antibody was coated overnight at 4° C. on a 96 well plate (200 ng/well) in 50 mM carbonate/bicarbonate buffer pH 9.5. Plates were washed in PBST then blocked for 2 hours at room temperature with 1% BSA in PBS. Serum (50 μl) was then added to the plate and incubated for 1 hour at room temperature. Plates were then washed six times with Phosphate buffered saline containing 0.05% Tween 20. Biotinylated 2D3 monoclonal antibody (2 ug/ml) in PBS containing 1% normal rabbit serum was then added and the plate further incubated at room temperature for 1 hour. After again washing six times, 50 μl of a 1:10000 dilution of streptavidin HRP in PBS containing 1% normal rabbit serum was added and the plate incubated at room temperature for 30 minutes at which time the plate was again washed six times. Development of signal with TMB substrate was as described above.

In the second assay 2D3 was coated on 96 well plates at 200 ng/well (Costar/Corning, Cambridge MA) overnight at 4° C. washed in PBST then blocked for two hours at room temperature. Serum (50 μl) was then added to the plate and incubated for 1 hour at room temperature. Plates were then washed six times with Phosphate buffered saline containing 0.05% Tween 20. Biotinylated 31A5 monoclonal antibody (0.5 μg/ml) in PBS containing 1% normal rabbit serum was then added and the plate further incubated at room temperature for 1 hour. After again washing six times the streptavidin-HRP and TMB incubations were performed as described above.

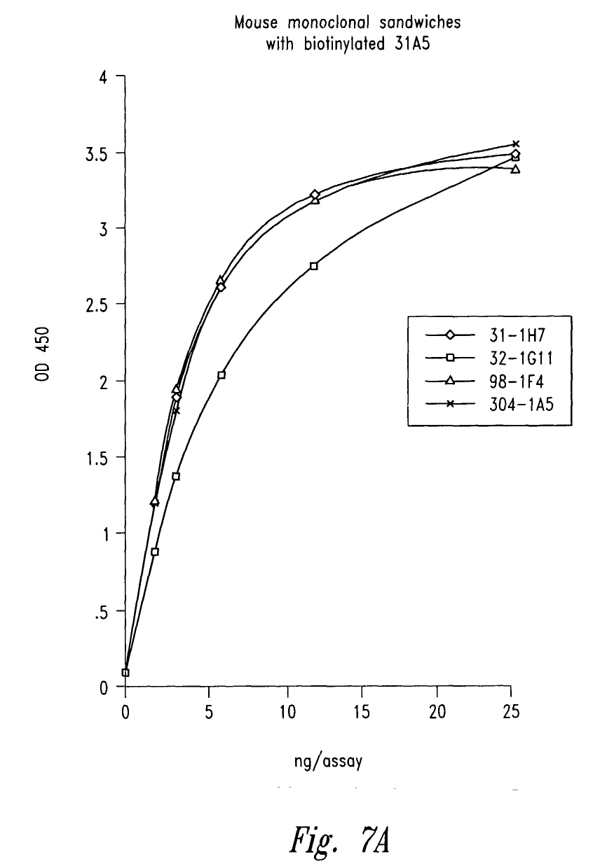

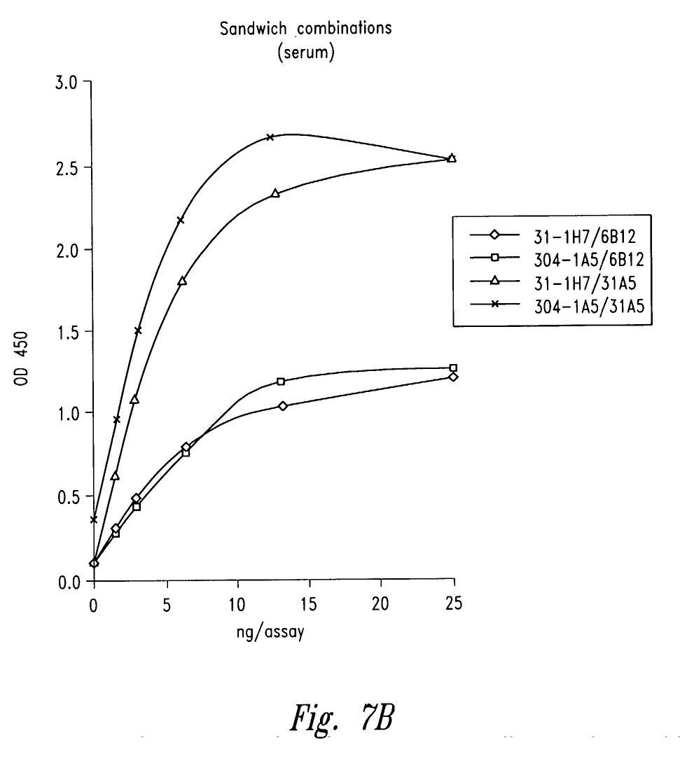

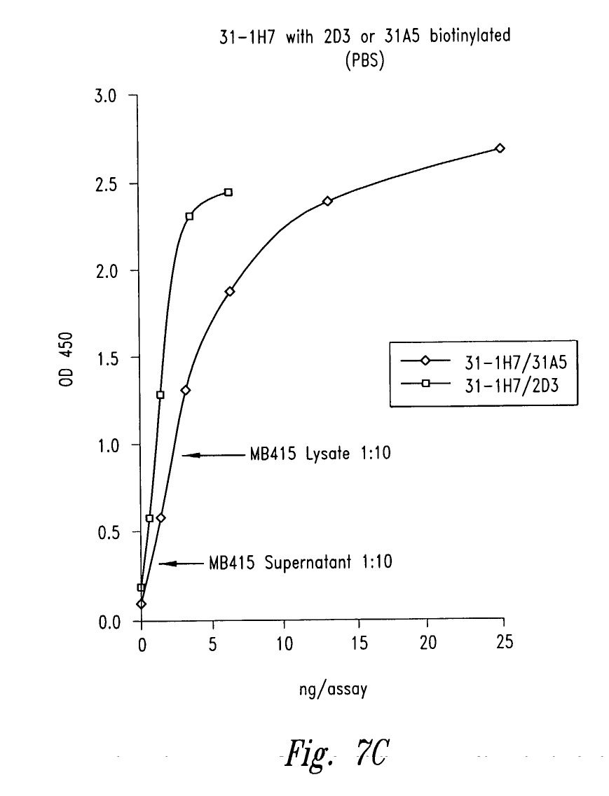

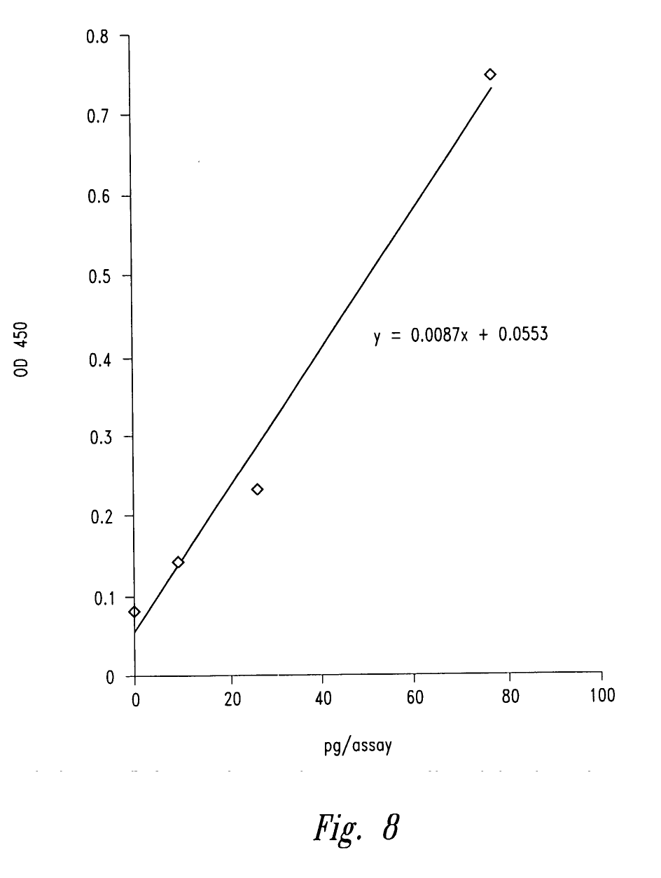

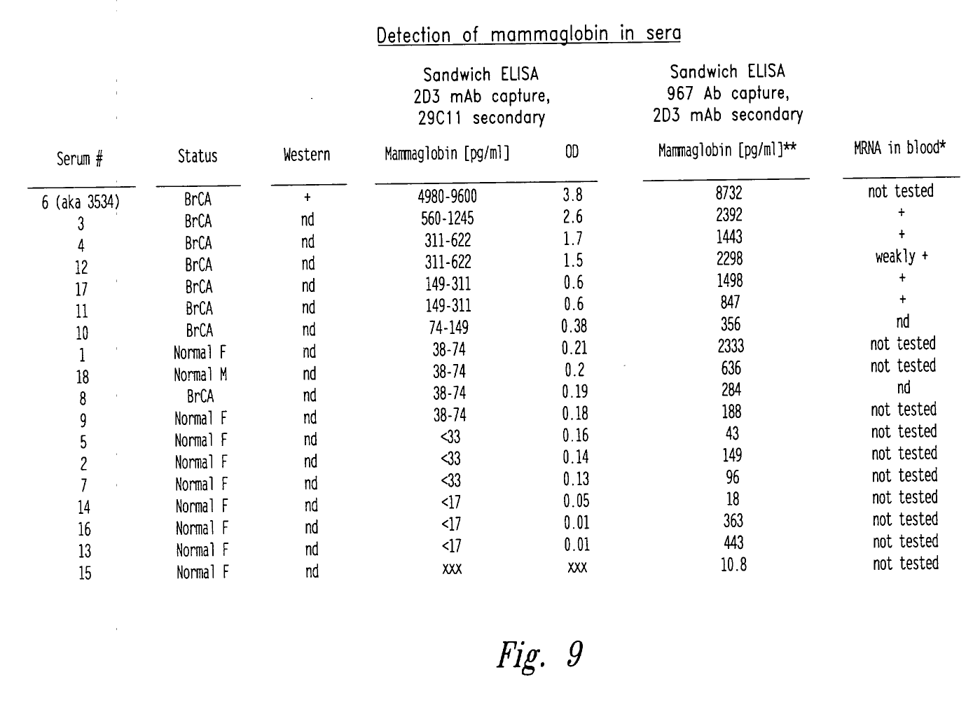

FIGS. 7A-7C are examples of the sandwiching of the mouse monoclonal antibodies with 31A5, 6B12 or 2D3 biotinylated and the ability to detect mammaglobin in MB415 lysates as well as supernatants. In FIG. 8 is shown the linear portion of the standard curve for the polyclonal anti-967 serum in combination with the monoclonal 2D3 biotinylated. This curve was used to quantitate mammaglobin serum samples of 7 patients with metastatic breast cancer. Of these 5/7 were positive for mammaglobin with serum levels in the 1-10 ng/ml range. In the same experiment 9/11 normal samples were negative and below the cut-off. Mammaglobin and mammaglobin RNA were detectable in these same breast cancer patient samples using a sandwich of 2D3 and 29C11 as shown in FIG. 9. For all experiments, mammaglobin levels were obtained using a standard curve, and negative and positive controls were as expected.

### Example 3

### Identification of Anti-Oligosaccharide Antibodies Specific for Mammaglobin

This Example illustrates the preparation of antibodies that specifically bind mammaglobin in a glycosylation-sensitive manner, including antibodies to differentially glycosylated sites of mammaglobin expressed in breast cancer cells.

An antibody library (Glycotech Corp., Rockville Md.) that encompasses 20-30 antibodies to different glycosylation combinations is screened using native mammaglobin from breast carcinoma cell lines via conventional ELISA and blotting techniques. Native mammaglobin is purified from MDA-MB-415 breast carcinoma cells using standard biochemical purification procedures. Both rabbits and mice are immunized with native mammaglobin. SLAM technology is used to develop rabbit monoclonal antibodies that bind to native mammaglobin but not to mammaglobin that has been stripped of oligosaccharides using deglycosylation enzymes. Identical approaches are used to screen hybridoma supernatants that are generated from mice immunized with native mammaglobin.

The glycosylation epitopes for the antibodies generated in rabbits and mice are mapped using a carbohydrate library (Glycotech Corp.). The carbohydrate library consists of a diverse array of oligosaccharide combinations permits the definition of carbohydrate epitopes to the antibodies. Upon identification, isolation and characterization of anti-oligosaccharide antibodies specific for mammaglobin, a sandwich ELISA assay is performed in which mammaglobin is captured from the sera of breast cancer patients with an anti-mammaglobin polyclonal or monoclonal antibody (which binds an epitope that is the 16 C-terminal amino acids of mammaglobin) and the anti-carbohydrate antibody is used for detection. The resulting ELISA assay provides sensitive and accurate diagnosis of breast cancer.

### Example 4

### Identification of Human CD4 T cell Epitopes for Mammaglobin

This Example illustrates the generation of CD4 T cells that recognize mammaglobin.

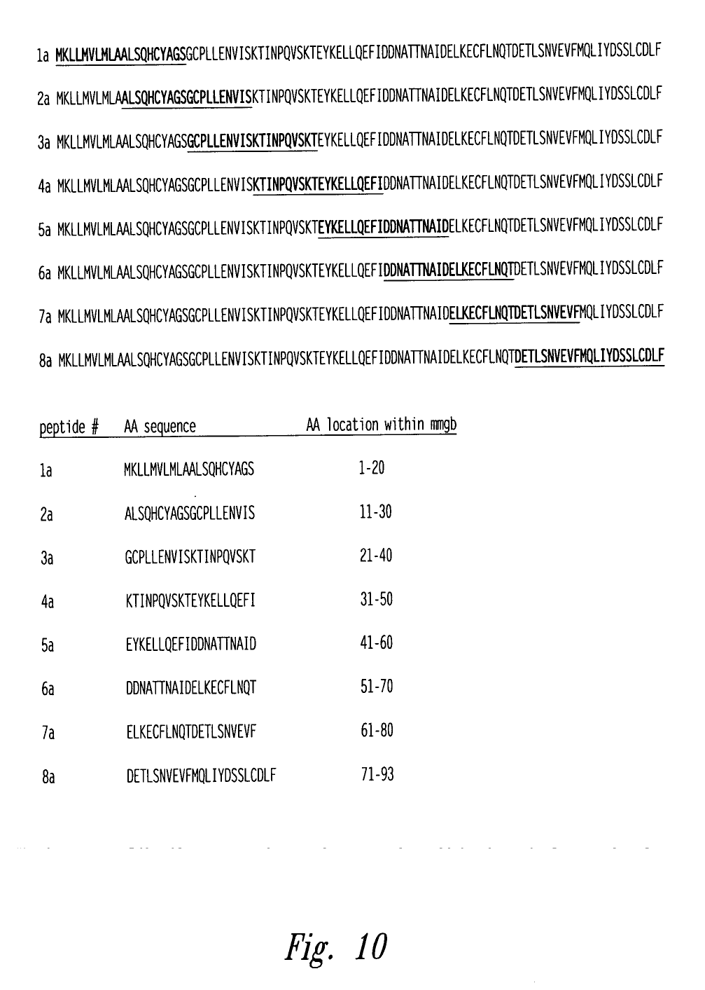

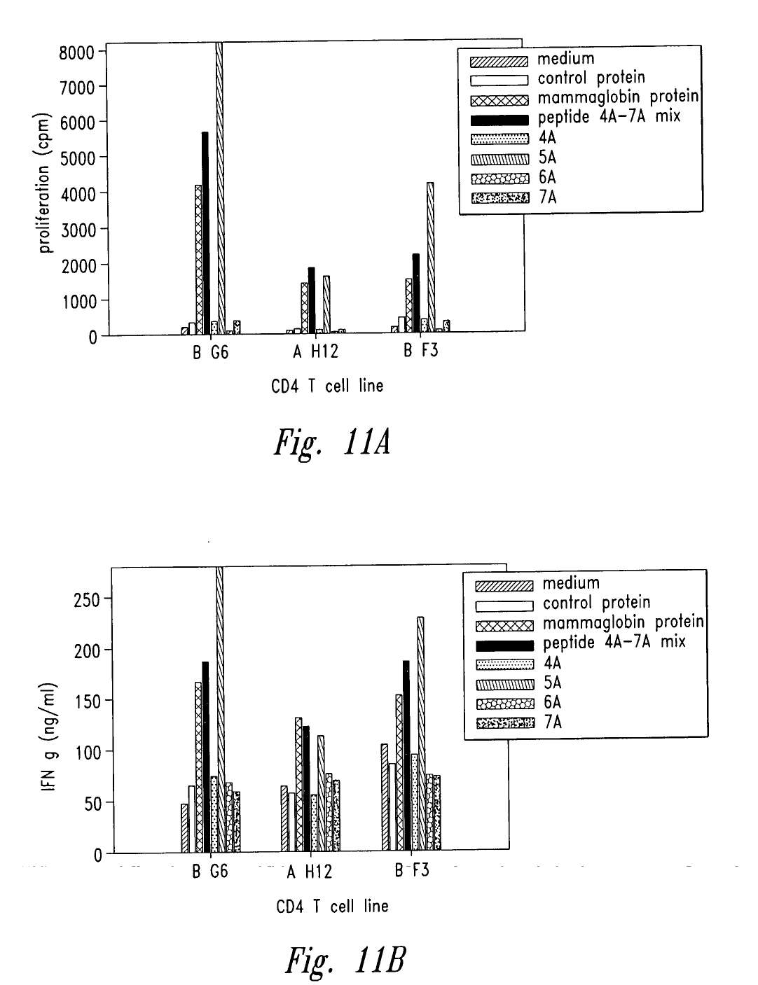

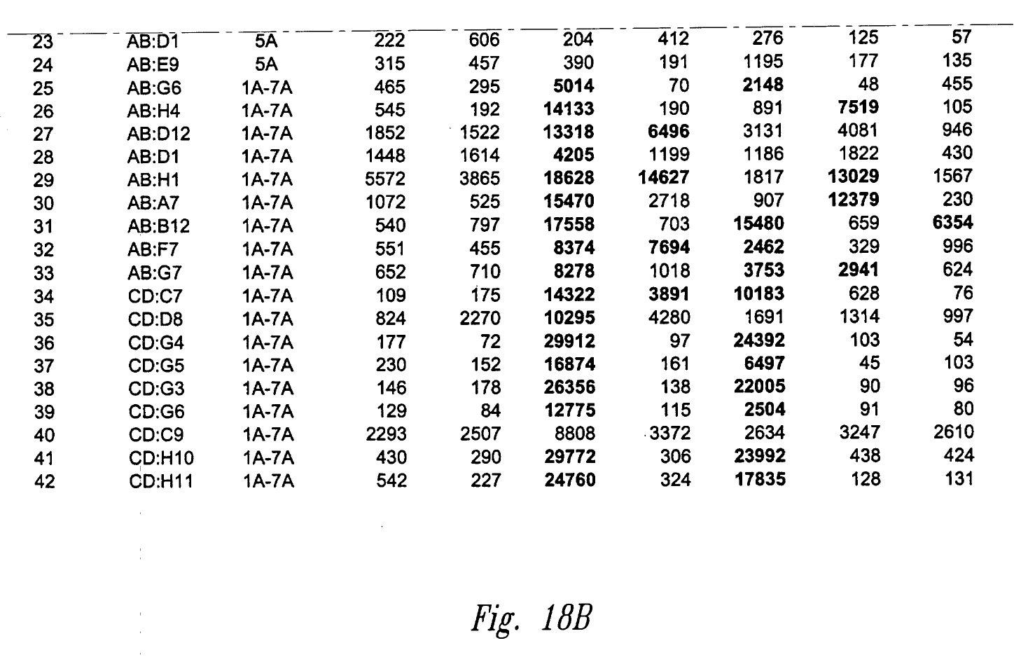

CD4 T cell responses were generated from PBMC of normal donors using dendritic cells (DC) pulsed with overlapping 20-mer peptides spanning the entire mammaglobin protein sequence. CD4⁺ T cells were stimulated 3-4 times with DC pulsed with a mixture of overlapping peptides (10 μg/mL each) in Iscove's modified Dulbecco's Medium (IMDM) containing IL-6 and IL-12 in the primary stimulation, and 0.5 ng/mL IL-2 and 5 ng/mL IL-7 in all other stimulations. The peptides are shown in FIG. 10. These lines were subsequently assayed for reactivity with the priming peptides or recombinant E. coli-derived mammaglobin. As shown in FIGS. 11A and 11B, a number of CD4 T cell lines demonstrated reactivity with the priming peptides as well as mammaglobin protein. The peptide specific reactivity of these lines was demonstrated in lines primed with peptides 3A (aa 21-40), 5A (aa 41-60) and 7A 9aa 61-80 of the mammaglobin protein sequence. The dominant reactivity of these lines appeared with peptide 5A (EYKELLQE-FIDDNATTNAID; SEQ ID NO: 4), corresponding to amino acids 41-60 of the mammaglobin sequence. These results indicate that peptide 5A represents an immunogenic CD4 epitope of mammaglobin. FIG. 18 shows the results of an assay for CD4⁺ proliferation.

### Example 5

### Identification of Human CD8 T Cell Epitopes for Mammaglobin

This Example illustrates the generation of CD8 T cells that recognize mammaglobin.

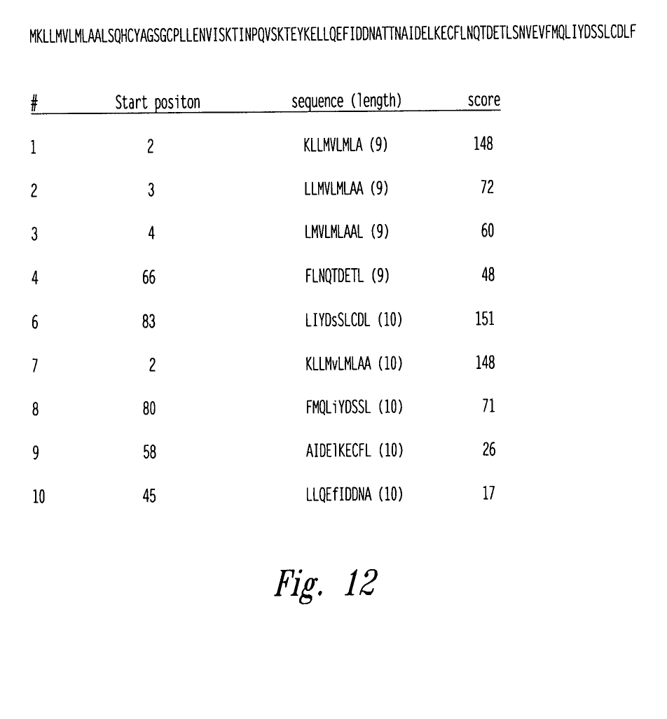

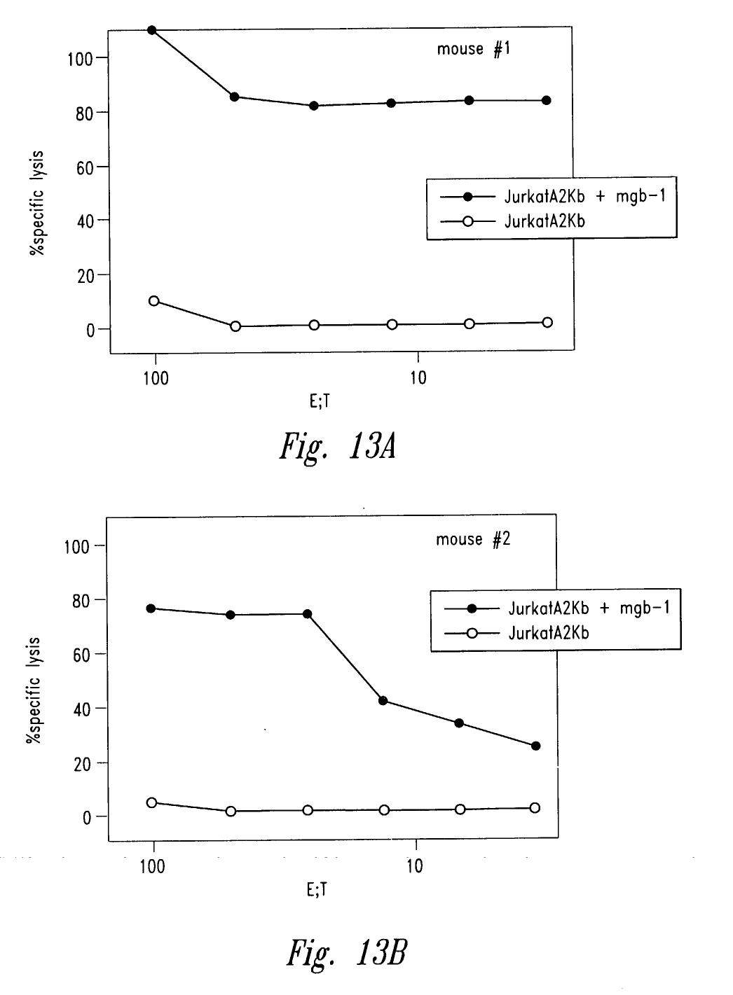

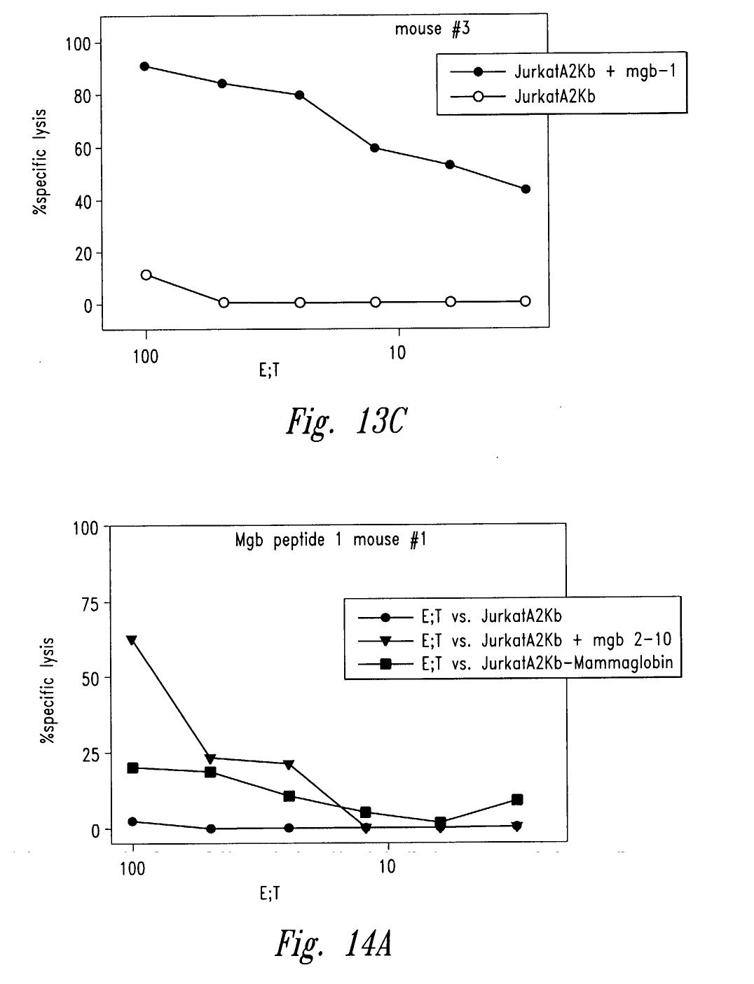

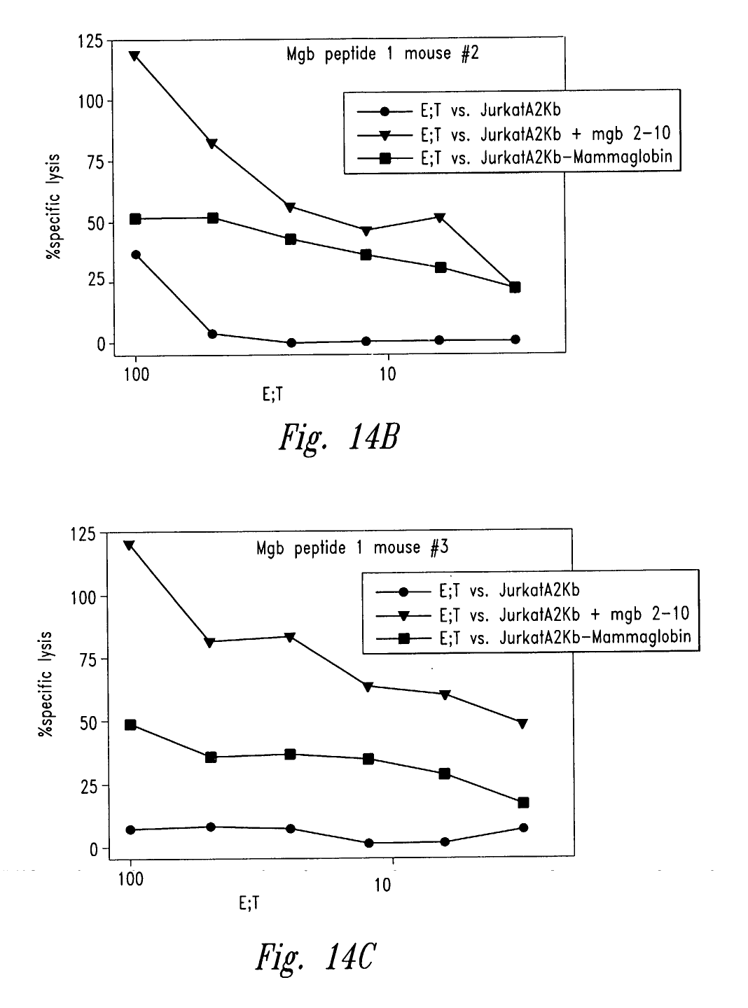

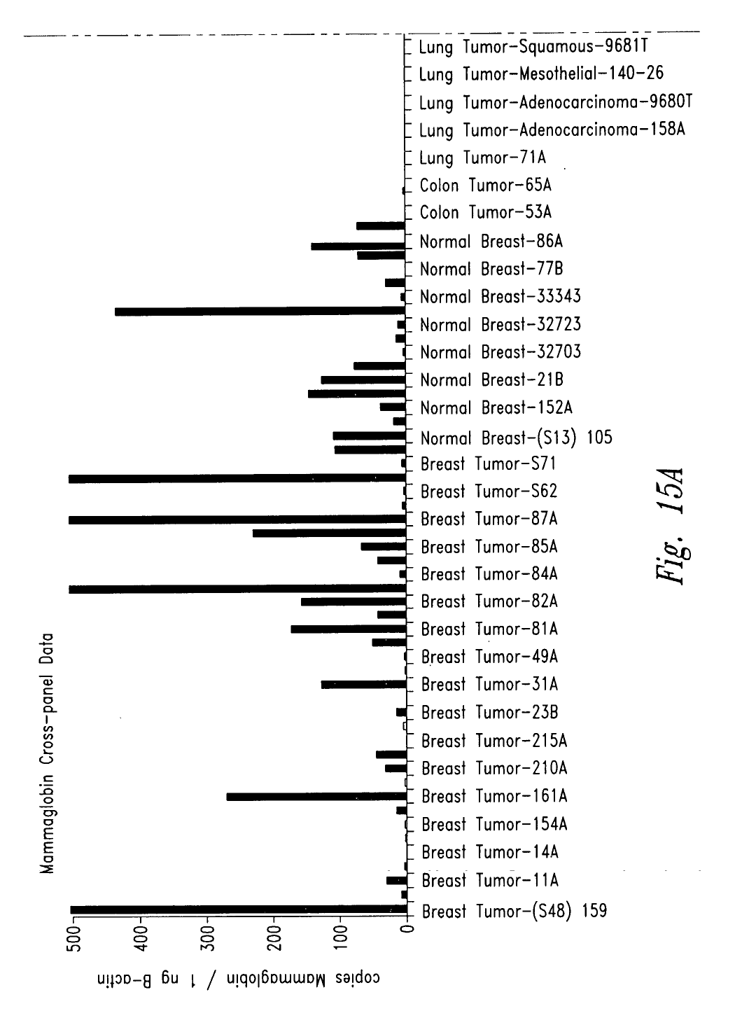

HLA A2 Kb mice were immunized with 9-mer peptides predicted to bind HLA A2 (shown in FIG. 12). Immunizations were performed subcutaneously in the footpad, using 100 μg of peptide together with 140 μg of hepatitis B virus core peptide (a Th peptide) in Freund's incomplete adjuvant. Three weeks post immunization, spleen cells were removed and cultured in vitro with peptide-pulsed APC to elicit CTL lines. CTL lines were subsequently evaluated for recognition of peptide-pulsed mammaglobin-transduced target cells in a standard chromium release assay. CTL lines recognizing peptide pulsed targets were then tested on targets transduced and stably expressing the mammaglobin protein. CTL lines from mice immunized with the 9-mer peptide KLLMVLMLA (mgb 1; SEQ ID NO: 5) corresponding to amino acids 2-10 of mammaglobin were shown to recognize both peptide-pulsed and mammaglobin transduced targets (FIGS. 13A-13C and 14A-14C). These data demonstrate that the 9-mer peptide KLLMVLMLA (SEQ ID NO: 5) is a naturally processed CTL epitope of mammaglobin, and is restricted by HLA A2.

### Example 6

### Detection of Mammaglobin RNA in Patient Blood Samples

This Example illustrates the use of PCR to detect mammaglobin expression in blood for the purpose of diagnosing breast cancer.

RNA extraction: RNA was extracted from frozen tumors and normal tissues and cell lines (MB415) as follows. Tissue samples were homogenized in Trizol reagent (Gibco, BRL) at 1 ml/50-100 mg of tissue using a homogenizer (Polytron) and cells mixed with Trizol reagent at 1 ml 5-10×10⁶ cells. The homogenized samples were then incubated at room temperature for 5 minutes followed by the addition of 0.2 ml of chloroform per 1 ml of Trizol reagent. Sample tubes were capped and vigorously shaken for 15 seconds followed by a further incubation at room temperature for 2-3 minutes. Samples were centrifuged at 12,000 g for 15 minutes at 2-8° C., and the upper aqueous phase was removed. The RNA preparation was transferred to a new tube and precipitated by addition of 0.5 ml isopropyl alcohol per 1 ml Trizol reagent used in the homogenation step. Samples were incubated at room temperature for 10 minutes, and then centrifuge for 10 minutes, 12,000 g at 2-8° C. The supernatant was removed from the gel like pellet and the pellet was washed once with 75% ethanol (1 ml/1 ml of Trizol). The sample was mixed and then centrifuged at 7,500 g for 5 minutes at 2-8° C. Supernatant was removed and the RNA pellet was briefly dried at room temperature and dissolved in RNase free water.

Isolated RNA was treated with DNase to remove any DNA contamination. The RNA (50 μg) in 75 μl nuclease free water and first strand buffer (Gibco BRL) was incubated with DNaseI (Ambion) in the presence of RNase inhibitor RNasin (Promega) at 37° C. for 30 minutes. The reaction mix was then precipitated with phenol/chloroform and centrifuged for 5 minutes in an eppendorf centrifuge maximum speed. The top layer was transferred to new tube, to which 20 μl 3M sodium acetate and 440 μl of 100% cold ethanol was added. The mixture was vortexed and spun again for 5 minutes. Supernatant was discarded and the pellet was washed with 75% cold ethanol and centrifuged. The RNA pellet was resuspended in RNase free water at 1-2 μg/ml.

RNA was extracted from whole blood using Dynal's Epithelial cell enrichment beads and Dynal's mRNA Direct kit (Dynal, Oslo, Norway) according to the manufacturer's instructions. RNA extracted via the Dynal extraction kit was immediately resuspended in 20 ml of Reverse transcription mix shown below and reverse transcribed.

Reverse Transcription: cDNA for use in real time PCR tissue panels was prepared as follows. 25 μg of RNA was incubated with 25 μl Oligo dT (Boehringer Mannheim) (100 ng/ml) at 70° C. for 10 minutes, and then with 125 μl of diluted reverse transcriptase buffer (Gibco, BRL containing 0.5 mM dNTP's 1000 units RNasin, 0.02 mM dithiothreitol and Superscipt II (Gibco BRL) at 42° C. for 1 hour. The reaction mix was then cooled to 4° C. for use in real-time PCR or frozen. The reaction mix for the epithelial extracted material was 20 μl of Superscript RT mix (4 ul of 5× buffer, 2 μl of 0.1 M DTT, 1 μl 10 mM dNTP mix, 1 μl (200 units) of superscript II and 12 μl of RNAse free water. The mix was then incubated at 50° C. for 5 minutes followed by 42° C. for 50 minutes then inactivated at 70° C. for 15 minutes.

Real Time PCR: Real time PCR analysis was performed on the Perkin Elmer/Applied Biosystems 7700 Prism instrument. Matching primers and fluorescent probes were designed for each of the genes of interest according to the primer express program provided by Perkin Elmer/Applied Biosystems (Foster City, Calif.). Primers and probes so produced can be used in the universal thermal cycling program in Real time PCR. Initially the primers and probes were titrated to determine the optimal concentrations using a checkerboard approach. A pool of cDNA from target tumors was used in this optimization process. These reagents were then used in Real Time PCR at their optimal concentrations. The reaction was performed in 25 μl volumes. In all cases the final probe concentration was 155 nM. dATP, dCTP and dGTP were at 0.2 mM and dUTP at 0.4 mM. Amplitaq gold and Amperase UNG (Perkin Elmer/Applied Biosystems, Foster City Calif.) were used at 0.625 units and 0.25 units per reaction. MgCl₂ was at a final concentration of 5 mM. Trace amounts of glycerol, gelatin and Tween 20 (Sigma Chem Co, St Louis, Mo.) were added to stabilize the reaction. Each reaction contained 2 μl of template. B-actin primers and probes were obtained from Perkin Elmer/Applied Biosystems (Foster City, Calif.) and used in a similar manner to quantitate the presence of B-actin in the samples. The forward primer was at 900 nM, reverse primer at 300 nM.

In order to quantitate the amount of specific RNA in the sample a standard curve was generated alongside using the plasmid containing the gene of interest. Standard curves were generated using the Ct values determined in the real-time PCR which are related to the initial cDNA concentration used in the assay, Standard dilutions ranging from 10-10⁶ copies of the gene of interest were used for this purpose. In addition, a standard curve was generated for β-actin ranging from 200 fg-2000 pg. This enabled standardization of initial RNA content of a tissue sample to the amount of β-actin for comparison purposes.

The primers and probes used were as shown in Table 1.

TABLE 1
Mammaglobin Primers and Probes

| | | SEQ |
|---|---|---|
| | Mammaglobin | ID NO: |
| Forward | TGCCATAGATGAATTGAAGGAATG | 8 |
| Primer | | |
| Reverse | TGTCATATATTAATTGCATAAACACCTCA | 9 |
| Primer | | |
| Probe | TCTTAACCAAACGGATGAAACTCTGAGCAATG | 10 |

The mammaglobin gene sequence contains three exons. Exon one spans bases 992 through 1110, exon two from 1713 through 1900, and exon three from 3789 through 3974. The start Met is at base 1056 and the stop codon is at base 3725. The primers and probes used for the quantitative real time PCR are located in exon 2; however, the reverse primer is divided between exon 2 and exon 3. The primer placement does not exclude amplification of genomic DNA. All tissue samples were DNase treated with Ambion DNase I. These samples were tested for the presence of contaminating DNA prior to use. RNA extracted from whole blood using Dynal's Epithelial cell enrichment beads and Dynal's mRNA Direct kit were not DNase treated, but this is a highly specific isolation method for RNA only.

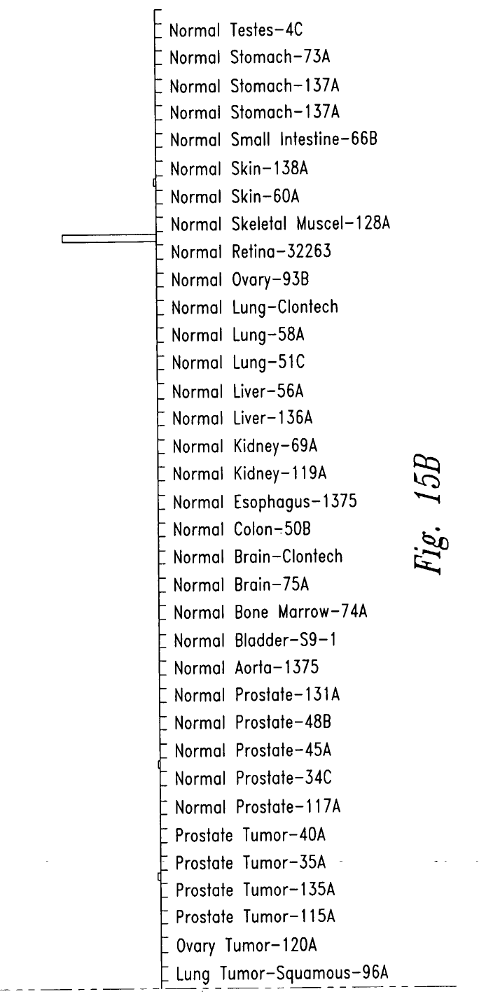

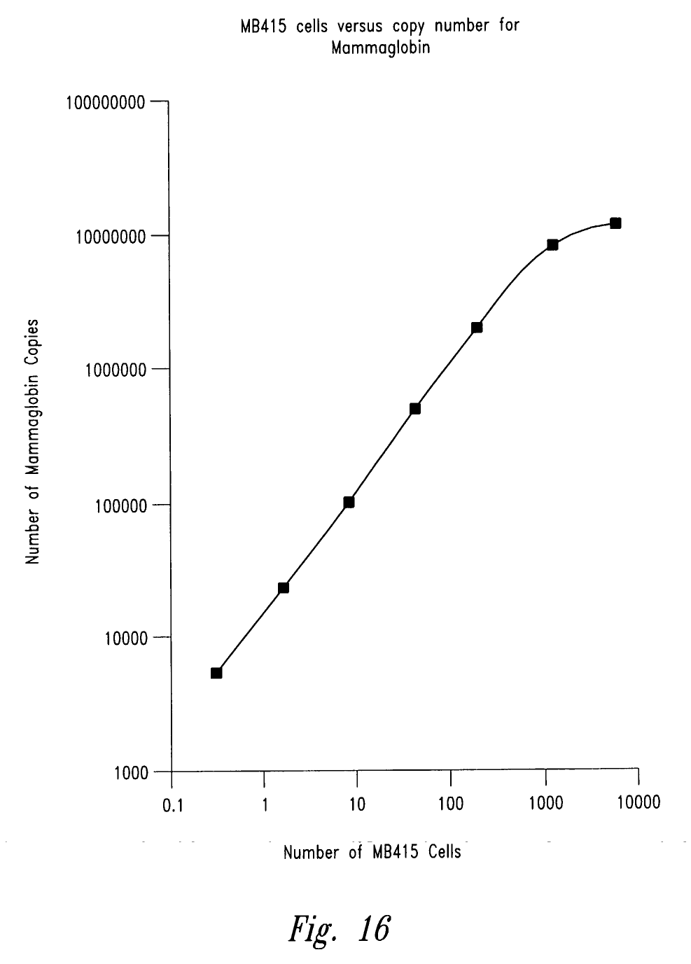

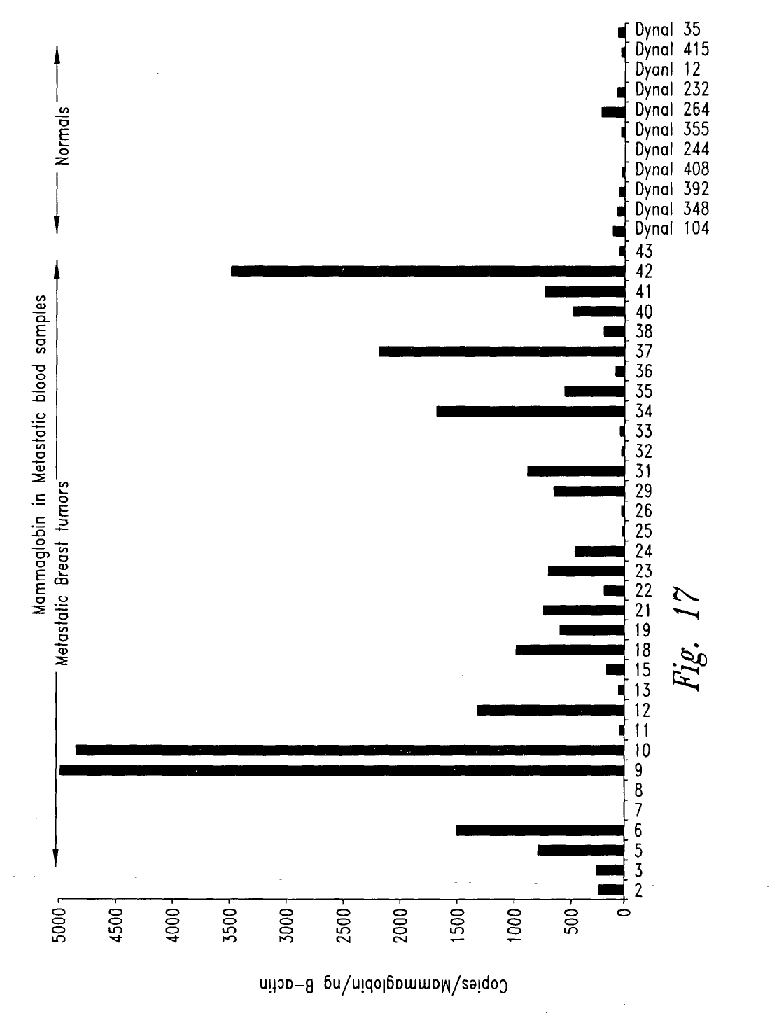

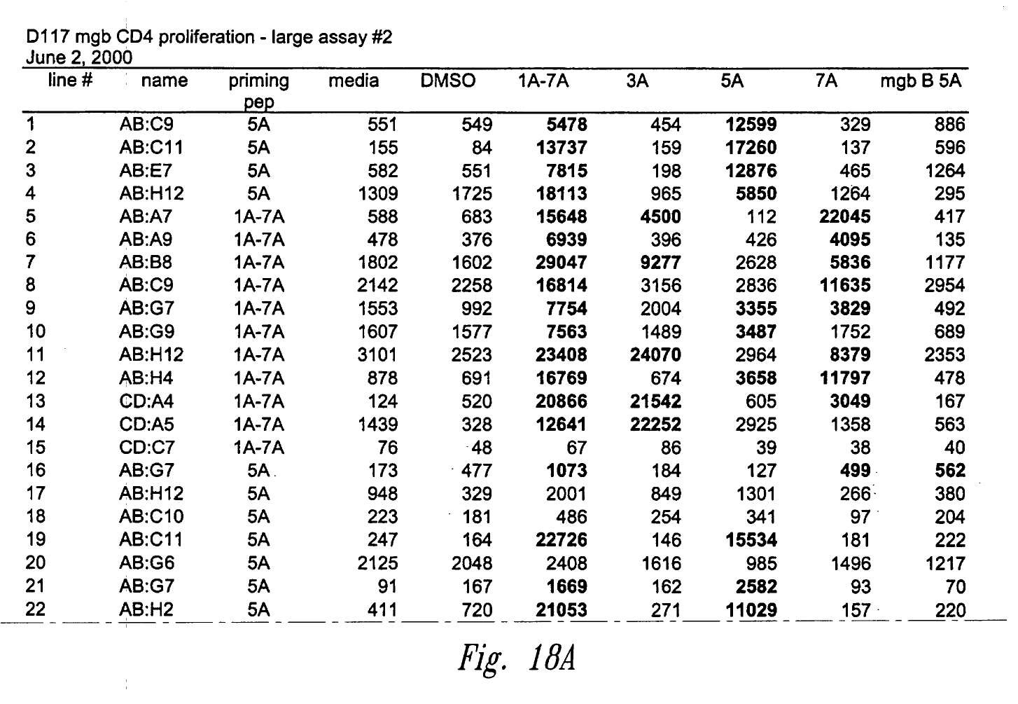

FIG. 15 shows the tissue distribution for mammaglobin showing the high degree of specificity for breast tissue. The skin sample shown to be positive was from a breast reduction. FIG. 16 shows the copies of mammaglobin message detectable in the breast cancer cell line MB415 as a function of the amount of cells indicating that one cell has 10000 copies. FIG. 17 shows the detection of mammaglobin in epithelial cells isolated, using the Dynal isolation method, from the peripheral blood of patients with metastatic breast cancer compared to similar isolates from normal blood samples. Thirty three metastatic and 11 normal samples were tested. The data indicate that mammaglobin can be detected in the blood of individuals with metastatic breast cancer, and that such detection may be used to diagnose the disease.

### Example 7

### Demonstration of Tumor Recognition by Mammaglobin-Specific CD4 T Cells

CD4⁺ T cells specific for mammaglobin were generated as described above. Briefly, CD4⁺ T cells were primed in vitro using dendritic cells and overlapping peptides spanning the entire mammaglobin protein sequence. Lines with peptide reactivity were screened for recognition of recombinant E. coli-derived mammaglobin protein. T cell lines demonstrating activity to recombinant mammaglobin were selected for further characterization.

To determine whether these CD4⁺ T cell lines were able to recognize native mammaglobin secreted from tumor cells or tumor cells expressing mammaglobin, the lines were tested for reactivity with the following:

recombinant E. coli derived mammaglobin

native mammaglobin purified from MB415 (mammaglobin+) breast tumor cells

deglycosylated native mammaglobin

denatured native mammaglobin

denatured, deglycosylated native mammaglobin

MB415 tumor cell lysate (mammaglobin+breast tumor cells)

SKOV3 tumor cell lysate (mammaglobin−ovarian tumor cells

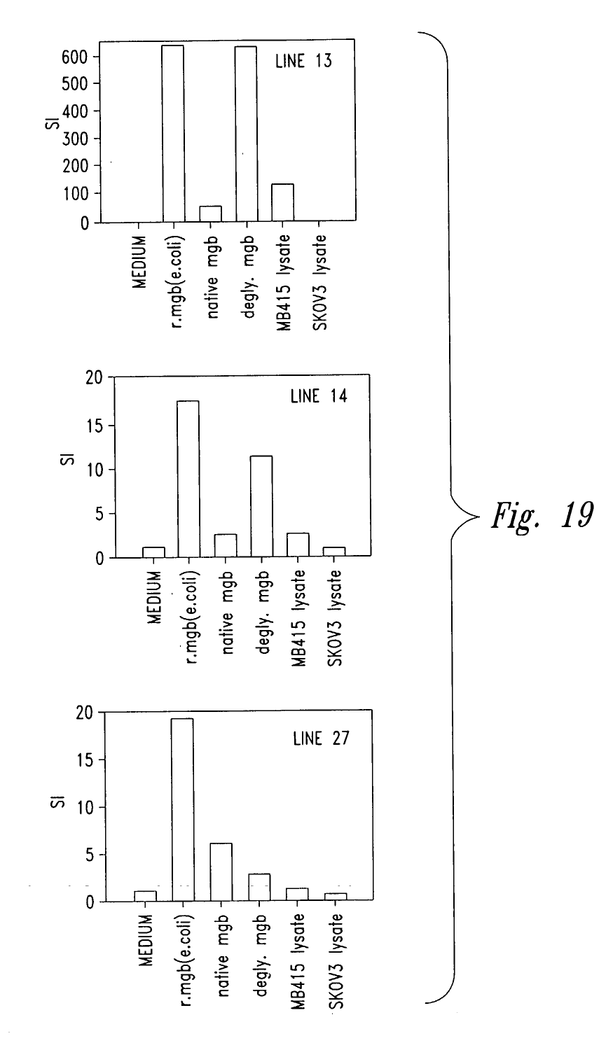

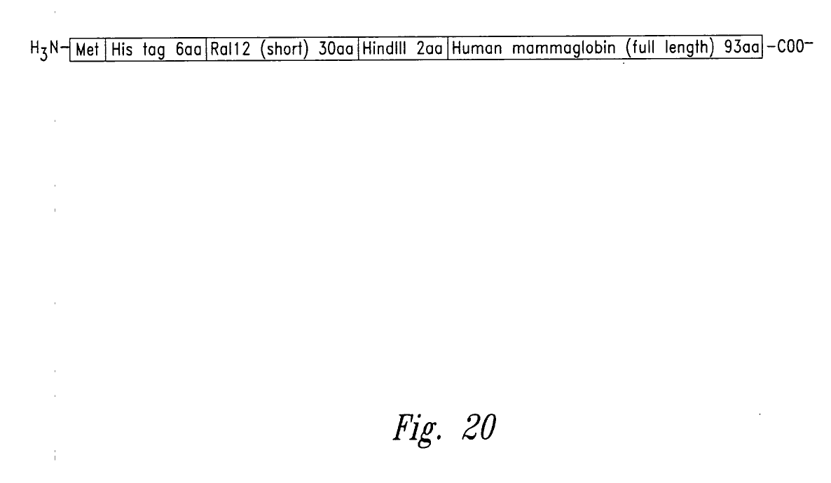

The results, shown in Table 2, demonstrate that lines #8 and #19 recognize native mammaglobin in the deglycosylated form. In addition, one line (line #13) recognizes native mammaglobin, and MB415 lysate as well as the deglycosylated forms of native mammaglobin demonstrating that this line recognizes mammaglobin expressed by tumor cells. In a subsequent assay (FIG. 19), 2 additional lines (#14 and #27) were also shown to recognize native mammaglobin and MB415 tumor cell lysate.

TABLE 2

| Reactivity with | Cell Line | | |
|---|---|---|---|
| Mammoglobin | Line 8* | Line 13 | Line 19 |
| Medium | 1 | 1 | 1 |
| Recombinant E. coli- | 145 | 375 | 300 |
| Derived | | | |
| Native | 2 | 50 | 1 |
| Denatured Native | 1 | 25 | 1 |
| Deglycosylated Native | 135 | 375 | 35 |
| Denatured, | 2 | 430 | 1 |
| Deglycosylated Native | | | |
| MB415 | 1 | 40 | 1 |
| SKOV3 | 2 | 0 | 1 |

*Values represent the stimulation index relative to the medium control.

The CD4 peptide epitope recognized by each of these lines is listed below in Table 3.

TABLE 3

| Line # | Peptide(s) Recognized |
|---|---|
| 13 | 3A, 7A |
| 8 | 7A |
| 19 | 5A |
| 14 | 3A, 5A, 7A |
| 27 | 3A |

In summary, these results demonstrate that mammaglobin-specific CD4 T cells can be generated in vitro by priming with peptides. Furthermore, some of these T cell lines demonstrate significant reactivity with mammaglobin-expressing tumor cells and/or mammaglobin purified from tumor cells.

### Example 8

### Expression of Recombinant Ra12 (s) MammFL, a Fusion Protein Consisting of Full Length Human Mammaglobin with Short Ra12

A Ra12(s) MammFL construct was engineered, consisting of short Ra12 linked to full length human mammaglobin. The fusion protein was produced in E. coli, and the recombinant protein is suitable for breast cancer therapy and diagnostics. The DNA sequence is shown in SEQ ID NO:46 and the protein sequence is shown in SEQ ID NO:47.

This example provides recombinant, Ra12(s)MammFL, which is an N-terminal Ra12 (short) fusion to full length human mammaglobin gene. The recombinant Ra12(s)MammFL is 132aa protein.

Recombinant versions of the mammaglobin gene have been made, but a full length version, including the N-terminal signal peptide, has not been made in an E. coli expression vector. Using hMamm in pcDNA 3.1, a midiprep (Wizard) was performed to obtain purified vector containing hMamm for use as template in PCR. Primers FLMamm1 (SEQ ID NO:48) and FLMamm2 (SEQ ID NO:49) were used to PCR amplify the full length gene, adding 5′ Hind III and 3′ Xho I sites for easy subcloning into pCRX1 vector. This subcloning strategy put the full length mammaglobin gene in frame at the 3′ end of the short form of Ra12. Vector and insert were digested with Hind III and Xho I. Vector was additionally treated with CIAP to prevent self-ligation. Ligation was done with pCRX1 and FLMamm at room temperature. Ligation mix was used ot transform NovaBlue E. coli host. Transformed E. coli was plated and grown overnight. Ten colonies were screened for insert by degestion with Hind III and Xho I. Positive clones were sequenced using T7 promoter and terminator primers. Sequencing confirmed pCRX1 Ra12(s)MammFL #1 (#61474). This clone was transformed into E. coli BLR (DE3) and HMS 174 (DE3) host strains for protein expression.

Cloning Primers:

Sense Primer—FLMamm1 #7769 FL Mamm 5′ primer, Hind III site

5′-gcgaagcttATGAAGTTGCTGATGGTCCTCATGC-3′ (SEQ ID NO:48)

full length 34 bp 50% 66C Tm

complement 25 bp 48% 58C Tm

Antisense Primer-FLMamm2 #7770 FL Mamm 3′ primer, Xhol site

5′-cggctcgagTTAAAATAAATCACAAAGACTGCTGTC-3′ (SEQ ID NO:49)

full length 36 bp 42% 63C Tm

complement 27 bp 30% 52C Tm

The Ra12(s)MammFL has the following characteristics—molecular weight of 14638.81 Daltons; 132 amino acids; isolectric point of 4.936, and charge at pH 7.0 of −8.033.

Protein Expression:

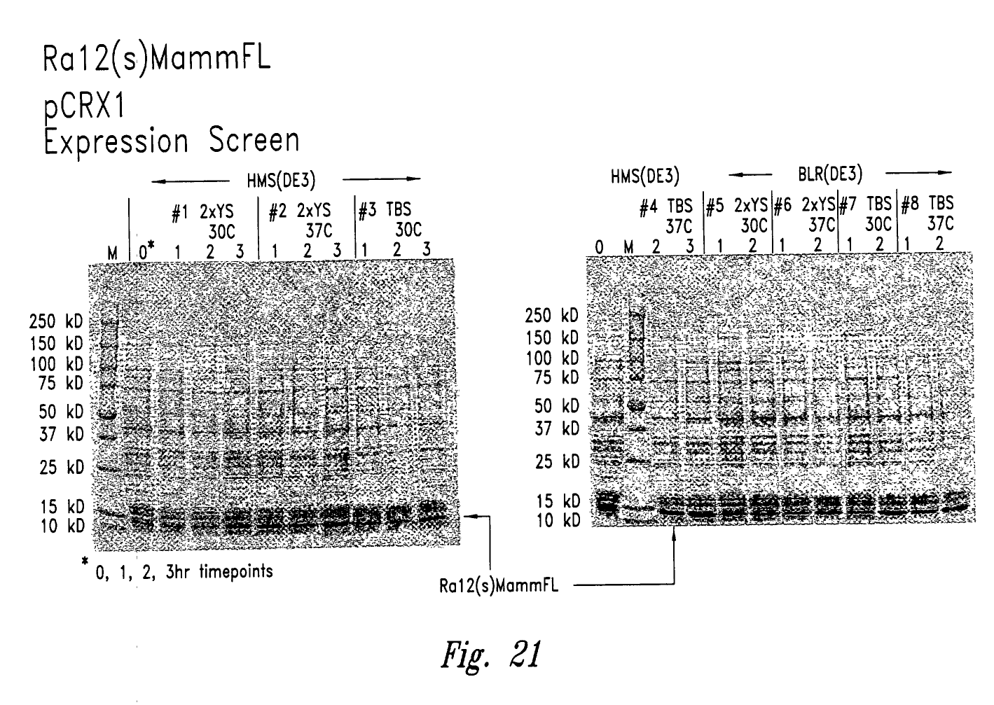

A standard “mini” expression screen was performed to determine the optimal induction conditions. BLR (DE3) cells grew slowly after inoculation, HMS 174 (DE3) grew faster, but both were induced achieving peak cell density only 1 hr after induction (though expression levels are highest 3 hr post-induction). Coomassie stained SDS-PAGE showed a specifically induced band at about 14 kD (FIG. 21). HMS 174 (DE3) strain grew the best, with optimal expression of Ra12(s)MammFL in TBS media at 37° C. induced for 3 hr.

### Example 9

### Identification of a Second, Naturally Processed HLA-A2 CTL Epitope for Mammaglobin

A second, HLA-A2 binding, naturally processed CTL epitope for mammaglobin was mapped. The cDNA and amino acid sequences of mammaglobin are set forth in SEQ ID NOs:69 and 27, respectively. To identify immunogenic and naturally processed CTL epitopes of mammaglobin, HLA-A2 Kb transgenic mice were immunized with recombinant adenovirus expressing mammaglobin. Following immunization, CTL lines were elicited by in vitro culture of spleen cells with mammaglobin transduced antigen presenting cells (APC). Immunization of HLA A2 Kb transgenic mice with recombinant adenovirus expressing mammaglobin elicited CTL that specifically recognize, in an HLA-A2 Kb restricted manner, target cells transduced with and stably expressing mammaglobin. However, these T cells failed to recognize the previously described HLA-A2 binding epitope (SEQ ID NO:5; amino acids 2-10 of mammaglobin) that was shown in Example 5 to be a naturally processed CTL epitope. To map the epitope recognized by these CTL, we used a transposon insertional mutation approach to disrupt translation of the antigenic mammaglobin protein expressed off a eukarytic vector, thereby creating 12 mammaglobin truncation mutants.

Transposon insertional mutagenesis was used herein as a means to identify the region of a cDNA that encodes a T cell epitope. More specifically, this was done by generating a panel of “mutant” polypeptides that were truncated as a result of random insertion of premature stop codons into the cDNA sequence that encodes the polypeptide/T cell antigen. DNA sequencing was used to locate the position of the individual insertions, in individual cloned plasmids, and a ranked panel of clones that encode progressively larger (or smaller) polypeptides were subsequently assayed for the presence of the epitope. Location of the epitope was then deduced from observing which clones, within the series, retained or lost the ability to stimulate T cells. Random insertion of stop codons into a polypeptide encoding cDNA was accomplished via a hyperactive form of Tn5 transposase in a reaction with supercoiled DNA and the modified transposon vector constructed for this purpose as described in further detail below.

Transposon vectors for use in this procedure are commercially available. Exemplary vectors include, but are not limited to, EZ::TN pMOD vector from Epicentre, (Madison, Wis.). It may be advantageous to add antibiotic resistance genes such as Kanamycin or Ampicillin resistance, to the vector. Conventional PCR methodologies may be used for this purpose using primers to clone the bacterial promoter and the gene for kanamycin drug resistance from a template such as pVR1012. The primers used herein contained the following elements: sequences corresponding to the first 15 bp of the 5′ promoter and the terminal 15 bp of the 3′ kanamycin coding region, stop codons in all six reading frames flanking this transcription unit and EcoRI 5′/PstI 3′ ends for cloning the final fragment into the transposon vector. The kanamycin gene enables selection of integrated transposons from a background of Amp starting plasmid. The stop codons are required for the cessation of translation regardless of which reading frame is utilized by the parent target plasmid and which direction the transposon integrated into the target relative to the cDNA Methionine initiating codon.

The transposon DNA used for insertional mutation was linearized and separated from the backbone by digestion with Pvu II (other appropriate restriction endonucleases may be used, depending on the backbone vector being used), and gel purified to obtain the 1.2 kb transposon fragment containing the above elements and a specific 19-basepair sequence from the backbone vector, (5′CTGTCTCTTATACACATCCT3′), at each end required for recognition by the Tn5 transposase enzyme. Equimolar ratios of the transposon and the target supercoiled plasmid were mixed with the buffer and Tn5 transposase as per manufacturers instructions. An aliquot of the reaction was used to transform E. coli which were then plated on kanamycin to allow growth of only transposon-containing clonal colonies. These colonies were individually picked and prepared for plasmid to be sequenced with primers originating just inside the transposon and moving 5′→3′ out into target sequence. Sequencing of these plasmids determined the insertion point and from a group of these sequences, a panel of plasmids was selected for T cell stimulation assays.

An alternative method was also used to screen for truncation mutants. In this method, rather than plating for individual colonies, a bulk culture was grown in media containing kanamycin. This bulk culture was prepared as one culture that contains clones whose transposon integrated randomly into either the cDNA or into the vector backbone and associated sequences (i.e., promoter, polyadenylation signal, etc.). DNA from this preparation was digested with restriction enzymes chosen to drop the cDNA and associated transposon from the vector. This resulted in four bands (two sets of two bands) when analyzed by gel electrophoresis: Vector and Vector with transposon (1.6 kb larger) and cDNA and “cDNA with transposon”. The “cDNA with transposon” was actually a population of cDNAs representing different insertional events. From this gel the vector alone and the cDNA with transposon bands were prepared for re-ligation and transformation. The resulting colonies plated on kanamycin were picked and prepared separately for sequencing, as above. Finally, to locate the area of the cDNA encoding the T cell epitope, plasmid DNAs from the ranked series of clones (which will ultimately represent translated proteins of greater or lesser length) were prepared for transfections. An appropriate antigen presenting cell was transfected and assayed for the ability to stimulated the T cells of interest as described in further detail below. This mutation approach can also be used for screening antibodies or other protein-protein interactions, by looking for retention or loss of binding/function.

The insertion mutants generated as described above were then tested for recognition by CTL as follows: HLA-A2 Kb expressing APC were transfected with ˜100 ng/well DNA of transposition mutants (see Table 4), or positive controls, and tested for stimulation of anti-mammaglobin T cells in a TNF assay. As a positive control, APC were transfected with clones of the mammaglobin, antigen positive, full-length cDNA construct.

By analysis of T cell recognition patterns and translation disruption (truncation) points, we were able to deduce the region of mammaglobin encoding the epitope. As shown in Table 4, T cells recognize mutant A1 transfectants, thus A1 did not interrupt translation of the T cell epitope. Transposition mutant D7 and all other mutants with truncations upstream of Al did interrupt translation of the T cell epitope as shown by the lack of T cell recognition of transfectants expressing these mutants. Thus, the epitope in mammaglobin maps to the C-terminal end of the protein, in the region between the insertion sites A1 and D7 (amino acids 78-93).

TABLE 4

| | Amino acid | T cell |
|---|---|---|
| Transposon Mutant | positions | response |
| A1 | 1-93 | + |
| E11 | 1-75 | − |
| C2 | 1-77 | − |
| D1 | 1-48 | − |
| D7 | 1-78 | − |
| All other mutants | | |
| Full-length | 1-93 | + |
| Mammaglobin | | |

Synthetic peptides representing this region were then assayed for stimulation of CTL to fine map the epitope to the minimal 9-mer. HLA-A2 kb expressing APC were incubated with titrated amounts of synthetic overlapping 9-mer peptides spanning the region between A1 and D7 (amino acids 78-93), and tested for stimulation of anti-mammaglobin T cells in a TNF assay. Jurkat/A2 kb cells transduced with full-length mammaglobin cDNA were used as a positive control. These data identified the 9-mer peptide, TLSNVECFM (SEQ ID NO:50), amino acids 73-81 of mammaglobin, as a second naturally processed, CTL epitope of mammaglobin presented in the context of HLA-A2 kb, and by extension HLA-A2, molecules.

### Example 10

### Expression of Full-Length Mammaglobin with C-terminal His Tag in E. Coli

Five different mammaglobin constructs were made for expression in an E. coli based expression system: full-length human mammaglobin with an N-terminal His tag; full-length human mammaglobin with a C-terminal His tag; mammaglobin variant B; mammaglobin variant A; full-length human mammaglobin with N and C-terminal His tags. The cDNA sequences for these constructs are set forth in SEQ ID NOs:51-55, respectively and the predicted amino acid sequences are set forth in SEQ ID NOs:59, 58, 57, 60, and 56, respectively. The constructs were made as described in detail below.

Construction of full-length hMAM with C-terminal His tag for production in E. coli: The full length coding region of human mammaglobin was PCR amplified with the following primers:

PDM-70 (SEQ ID NO:61) 5′ cttgatcatatgaagttgctgatggtcctc 3′ Tm 66° C.

PDM-71 (SEQ ID NO:62) 5′ ccgccgaattcatcaaaataaatcacaaagactgctgtc 3′ Tm 64° C.

The PCR conditions were as follows: 10 μl 10× Pfu buffer, 1 μl 10 mM dNTPs, 2 μl 10 μM each oligo, 83 μl sterile water, 1.5 μl Pfu DNA polymerase (Stratagene, La Jolla, Calif.), 50 ng DNA. The reaction was initially denatured for 2 minutes at 96° C., followed by 40 cycles of 96° C. for 20 seconds, 64° C. for 15 seconds and 72° C. for 45 seconds, followed by a final extension of 72° C. for 4 minutes. The PCR product was digested with NdeI and EcoRI and cloned into pET28, which was digested with NdeI and EcoRI. The correct clone was determined through sequence analysis and then transformed into BL21 (DE3) CodonPlus cells for expression.

Construction of full-length hMAM with N-terminal His tag for production in E. coli: The full length coding region of hMAM was PCR amplified with the following primers:

PDM-551 (SEQ ID NO:63) 5′ cacatgaagttgctgatggtcctcatgctggc 3′ Tm 66° C.

PDM-491 (SEQ ID NO:64) 5′ ccgccgaattcatcaaaataaatcacaaagactgctgtc 3′ Tm 64° C.

The PCR conditions were as follows: 10 μl 10× Pfu buffer, 1 μl 10 mM dNTPs, 2 μl 10 μM each oligo, 83 μl sterile water, 1.5 μl Pfu DNA polymerase (Stratagene, La Jolla, Calif.), 50 ng DNA. The reaction was initially denatured for 2 minutes at 96° C., followed by 40 cycles of 96° C. for 20 seconds, 64° C. for 15 seconds, 72° C. for 45 seconds, followed by a final extension of 72° C. for 4 minutes. The PCR product was digested with EcoRI and cloned into pPDM His, which was digested with Eco72I and EcoRI. The correct clone was determined through sequence analysis and then transformed into BL21 (DE3) CodonPlus cells for expression.

Construction of full-length hMAM for production in E. coli: The mature coding region of hMAM was PCR amplified with the following primers

(hMAM MAT A):

PDM-485 (SEQ ID NO:65) 5′ cctattatcatatgggctctggctgccccttattg 3′ Tm 66° C.

PDM-491 (SEQ ID NO:64) 5′ ccgccgaattcatcaaaataaatcacaaagactgctgtc 3′ Tm 65° C.

The mature coding region plus some of the leader sequence of hMAM was PCR amplified with the following primers (hMAM MAT B):

PDM-486 (SEQ ID NO:66) 5′ cctattatcatatgcactgctacgcaggctctgg 3′ Tm 65° C.

PDM-491 (SEQ ID NO:64) 5′ ccgccgaattcatcaaaataaatcacaaagactgctgtc 3′ Tm 65° C.

The PCR conditions were as follows: 10 μl 10× Pfu buffer, 1 μl 10 mM dNTPs, 2 μl 10 μM each oligo, 83 μl sterile water, 1.5 μl Pfu DNA polymerase (Stratagene, La Jolla, Calif.), 50 ng DNA. The reaction was initially denatured for 2 minutes at 96° C., followed by 40 cycles of 96° C. for 20 seconds, 64° C. for 15 seconds, 72° C. for 45 seconds, followed by a final extension of 72° C. for 4 minutes. The PCR products were digested with NdeI and EcoRI and cloned into pPDM His, which was digested with NdeI and EcoRI. The correct clone was determined through sequence analysis and then transformed into BL21 (DE3) CodonPlus cells for expression.

Construction of full-length hMAM with no leader sequence with 2 His tags in pPDM: The Mammoglobin coding region was PCR amplified with the following primers:

PDM-343 (SEQ ID NO:67) 5′ cactgctacgcaggctctgg 3′ Tm 56° C.

PDM-342 (SEQ ID NO:68) 5′ ggtgctcgagattatcagccgcc 3′ Tm 66° C.

The PCR conditions were as follows: 10 μl 10× Pfu buffer, 1 μl 10 mM dNTPs, 2 μl 10 μM each oligo, 83 μl sterile water, 1.5 μl Pfu DNA polymerase (Stratagene, La Jolla, Calif.), 0.5 ng DNA. The reaction was initially denatured for 2 minutes at 96° C., followed by 40 cycles of 96° C. for 20 seconds, 64° C. for 15 seconds, 72° C. for 45 seconds, followed by a final extension of 72° C. for 4 minutes. The PCR product was then digested with XhoI (NEB, Beverly, Mass.). This was then cloned into the pPDM His (a modified pET28 vector with a His tag in frame on the 5′ end) which was cut with Eco72I and XhoI. The construct with the correct sequence was then transformed into BL21 (DE3) pLysS and AD494 (DE3) pLysS cells.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. An isolated polypeptide comprising at least 7 consecutive amino acid residues of human mammaglobin, wherein the consecutive amino acid residues are present within a sequence selected from the group consisting of amino acids 21-40 of SEQ ID NO:27, amino acids 61-80 of SEQ ID NO:27, and the amino acid sequence set forth in SEQ ID NO:50, wherein no more than 30 consecutive residues of human mammaglobin are present within the polypeptide and wherein one or more mammaglobin-specific T cells specifically reacts with said polypeptide.

2. The polypeptide of claim 1 wherein the polypeptide comprises at least 15 consecutive amino acid residues of human mammaglobin.

3. A composition comprising a polypeptide according to claim 1, in combination with a physiologically acceptable carrier.

4. A composition comprising a polypeptide according to claim 1, in combination with an immunostimulant.

5. The composition of claim 4 wherein the immunostimulant is an adjuvant.

6. A method for inhibiting the development of breast cancer in a patient, comprising administering to a patient an effective amount of the composition according to claim 4, and thereby inhibiting the development of breast cancer in the patient.

7. A diagnostic kit, comprising:
(a) one or more polypeptides according to claim 1; and
(b) a detection reagent comprising a reporter group.

8. The kit of claim 7 wherein the polypeptides are immobilized on a solid support.

9. The kit of claim 8 wherein the solid support comprises nitrocellulose, latex or a plastic material.

10. The kit of claim 7 wherein the detection reagent comprises an immunoglobulin, anti-immunoglobulin, protein G, protein A or lectin.

11. The kit of claim 7 wherein the reporter group is selected from the group consisting of radioisotopes, fluorescent groups, luminescent groups, enzymes, biotin and dye particles.

12. A method for removing tumor cells from a biological sample, comprising contacting a biological sample with T cells that specifically react with a polypeptide of claim 1, wherein the step of contacting is performed under conditions and for a time sufficient to permit the removal of cells expressing mammaglobin or a peptide epitope thereof from the sample.

13. The method of claim 12, wherein the biological sample is blood or a fraction thereof.

14. A method for inhibiting the development of breast cancer in a patient, comprising administering to a patient a biological sample treated according to the method of claim 12.

15. A method for stimulating and/or expanding T cells specific for mammaglobin, comprising contacting T cells with a peptide according to claim 1, wherein the contact is performed under conditions and for a time sufficient to permit stimulation and/or expansion of T cells.

16. An isolated T cell population, comprising T cells prepared according to the method of claim 15.

17. A method for inhibiting the development of breast cancer in a patient, comprising administering to a patient an effective amount of a T cell population according to claim 16.

18. A method for inhibiting the development of breast cancer in a patient, comprising the steps of:
(a) incubating CD4⁺ and/or CD8⁺ T cells isolated from a patient with a polypeptide according to claim 1, such that T cells proliferate; and
(b) administering to the patient an effective amount of the proliferated T cells, and thereby inhibiting the development of breast cancer in the patient.

19. A method for inhibiting the development of breast cancer in a patient, comprising the steps of:
(a) incubating CD4⁺ and/or CD8⁺ T cells isolated from a patient with a polypeptide according to claim 1, such that T cells proliferate;
(b) cloning at least one proliferated cell; and
(c) administering to the patient an effective amount of the cloned T cells, and thereby inhibiting the development of breast cancer in the patient.

20. A composition comprising a polynucleotide selected from the group consisting of SEQ ID NOs:46 and 51-55 in combination with an immunostimulant.

21. The composition of claim 20 wherein the immunostimulant is an adjuvant.

22. A composition comprising a polypeptide selected from the group consisting of SEQ ID NOs:47 and 56-60 in combination with an immunostimulant.

23. The composition of claim 22 wherein the immunostimulant is an adjuvant.


SEQUENCE LISTING (69 sequences)

SEQ ID NO: 1 - protein, 20 aa, Homo sapien
IDELKECFLNQTDETLSNVE

SEQ ID NO: 2 - protein, 15 aa, Homo sapien
TTNAIDELKECFLNQ

SEQ ID NO: 3 - protein, 21 aa, Homo sapien
SQHCYAGSGCPLLENVISKTI

SEQ ID NO: 4 - protein, 20 aa, Homo sapien
EYKELLQEFIDDNATTNAID

SEQ ID NO: 5 - protein, 9 aa, Homo sapien
KLLMVLMLA

SEQ ID NO: 6 - protein, 13 aa, Homo sapien
QEFIDDNATTNAI

SEQ ID NO: 7 - protein, 13 aa, Homo sapien
LKECFLNQTDETL

SEQ ID NO: 8 - DNA, 24 bp, Artificial Sequence
  PCR primer
tgccatagatgaattgaaggaatg

SEQ ID NO: 9 - DNA, 29 bp, Artificial Sequence
  PCR primer
tgtcatatattaattgcataaacacctca

SEQ ID NO: 10 - DNA, 32 bp, Artificial Sequence
  Probe
tcttaaccaaacggatgaaactctgagcaatg

SEQ ID NO: 11 - protein, 20 aa, Oryctolagus cuniculus
IDELKECFLNQTDETLSNVE

SEQ ID NO: 12 - protein, 20 aa, Oryctolagus cuniculus
ELLQEFIDDNATTNAIDELK

SEQ ID NO: 13 - protein, 15 aa, Oryctolagus cuniculus
TTNAIDELKECFLNQ

SEQ ID NO: 14 - protein, 20 aa, Oryctolagus cuniculus
ELLQEFIDDNATTNAIDELK

SEQ ID NO: 15 - protein, 20 aa, Oryctolagus cuniculus
ELLQEFIDDNATTNAIDELK

SEQ ID NO: 16 - protein, 21 aa, Oryctolagus cuniculus
SQHCYAGSGCPLLENVISKTI

SEQ ID NO: 17 - protein, 20 aa, Oryctolagus cuniculus
ELLQEFIDDNATTNAIDELK

SEQ ID NO: 18 - protein, 21 aa, Mus musculus
SQHCYAGSGCPLLENVISKTI

SEQ ID NO: 19 - DNA, 405 bp, Oryctolagus cuniculus
caccatggagacaggcctgcgctggcttctcctggtcgctgtgctcaaaggtgtccagtgtcagtcgctggaggagtccggcggtcgcctggtaacgcctggaggatccctgacactcacctgcacagtctctggaatcgacctcagtagctatggagtgggctgggtccgccaggctccagggaaggggctggaatacatcggaatcattagtaaaattgataacacatactacgcgaactgggcgaaaggccgattcaccatctccaaaacctcgtcgaccacggtggatctgaaaatgaccagtctgacaaccgaggacacggccacctatttctgtaccagagggtcttttgatccctggggcccaggcaccctggtcaccgtctcctcagggcaacctaa

SEQ ID NO: 20 - DNA, 414 bp, Oryctolagus cuniculus
caccatggagacaggcctgcgctggcttctcctggtcgctgtgctcaaaggtgtccagtgtcagtcggtggaggagtccgggggtcgcctggtcacgcctgggacacccctgacactcacctgcacagtctctggattctccctcagcagctacgacatgacctgggtccgccaggctccagggaaggggctggaatggatcggaaccattagtactattggtagcccattttacgcgagctgggcgagaggccgattcaccatctccaaaacctcgaccacggtggatctgaaaatcaccaatccgacaaccgaggacacggccacgtatttttgcggcagatttcggattgctggtgatggtgccttctggggcccaggcacgctggtcaccgtctcctcagggcaacctaa

SEQ ID NO: 21 - DNA, 414 bp, Oryctolagus cuniculus
caccatggagacaggcctgcgctggcttctcctggtcgctgtgctcaaaggtgtccagtgtcagtcggtggaggagtccgggggtcgcctggtcacgcctaggacacccctgacactcacctgcacagtctctggattctccctcagcagctacgacatgacctgggtccgccaggctccagggaaggggctggaatggatcggaaccattagtactattggtagcccattttacgcgacctgggcgagaggccgattcaccatctccaaaacctcgaccacggtggatctgaaaatcaccaatccgacaaccgaggacacggccacgtatttttgcggcagatttcggattgctggtgatggtgccttctggggcccaggcacgctggtcaccgtctcctcagggcaacctaa

SEQ ID NO: 22 - DNA, 414 bp, Oryctolagus cuniculus
caccatggagacaggcctgcgctggcttctcctggtcgctgtgctcaaaggtgtccggtgtcagtcggtggaggagtccgggggtcgcctggtcacgcctgggacacccctgagattcacctgcacagtctctggaatcgacctcagcacctacgacatgacctgggtccgccaggctccagggaagggactggaatggatcggaaccattagtactcttggtacccctttttccgccaattgggcgagaggccgattcaccatctccaagacctcgaccacggtggatctgaaaatcgccagtccgacgaccgaagacactgccacatatttttgtggcagattgcggattgctcatgatggtgccttctggggcccaggcacgctggtcaccgtctcctcagggcaacctaa

SEQ ID NO: 23 - DNA, 422 bp, Oryctolagus cuniculus
cccatggagacaggcctgcgctggcttctcctggtcgctgtgctcaaaggtgtccagtgtcaggagcagctgaaggagtccggaggaggcctggtcacgcctgggacacccctgacactcacctgcacagtgtctggaatcgacctcaatatcgatgcaatgagctgggtccgccaggctccagggaaggggctggaatggatcggaattattggtactcgtggtggcacatggttcgcgagctgggcgaaaggccgattcaccatctccaaaaccccgaccacagtggatctgaaaatccccagtccgacaaccgaggacacggccacctatttctgtgccagtatctattctgatagtggtacttatacgaccttgtggggcccaggcaccccggtcaccgtctcctcagggcaacctaa

SEQ ID NO: 24 - DNA, 414 bp, Oryctolagus cuniculus
caccatggagacaggcctgcgctggcttctcctggtcgctgtgctcaaaggtgtccagtgtcagtcggtggaggagtccgggggtcgcctggtcacgcctgggacacccctgacactcacctgcaccgtctctggattctccctcagcagcgtcgacatgacctgggtccgccaggctccagggaaggggctggaatggatcggaaccattagtactcgtagtagcacatactacgcgagctgggcgaaaggccgattcaccatctccaaaacctcgaccacggtggatctgaaaatcaccagtccgacaaccgaggacacggccacgtatttctgtggcagatttcggattgctggtgatggtgccttctggggcccaggcacgctggtcaccgtctcctcagggcaacctaa

SEQ ID NO: 25 - DNA, 412 bp, Oryctolagus cuniculus
  misc_feature 76, 104, 106, 228, 352, 395: n = A,T,C or G
ggaaggctgcgctggcttttcctggtcgctgtgctcagaggtgtccagtgtcagtcgctggaggagtccgggggtngcctggtaacgcctgggacacccctganantcacctgcacagcctttggattttccctcagtagctggtcaatgagctgggtccgccaggctccagggaaggggctggaatggatcggaatgattggtattgttggtagtggcacataatangcgacctgggcgaaaggccgattcaccatttccaaaaccttgtgaccacggtcgatttgaaaatgaccagtttgacaaccgaggacacggccacctatttttgtgtcagagggggtagttttanttttgctaccgccttgtggggcccaggcaccctggtcaccgtntcctcagggcaacctaa

SEQ ID NO: 26 - DNA, 402 bp, Oryctolagus cuniculus
  misc_feature 75, 86, 105, 119, 140, 146, 191, 205, 385: n = A,T,C or G
ttgcaggctgcgtggttttcctggtcgctgtgctcaaaggtgtccagtgtcagtcggtggaggagtccgggggtngcctggtaacncctgggacacccctgacanttttttgcaaagtntttggattttccctcagcagntacganatgacctgggtccgccaggctccagggaaggggctggaatggatnggaaccattagtanttgtggtaatggataatacgcgacctgggcgaaaggccgattcaccatttccaaaaccttgaccaccgtggatttgaaaatcaccagtccgacaaccgaggacacggccaagtatttttgtggcagatttcggattgctggtgatggtgcttttggggcccgggcacgctggtcaccgtntcctcagggcaacctaa

SEQ ID NO: 27 - protein, 93 aa, Homo sapien
MKLLMVLMLAALSQHCYAGSGCPLLENVISKTINPQVSKTEYKELLQEFIDDNATTNAIDELKECFLNQTDETLSNVEVFMQLIYDSSLCDLF

SEQ ID NO: 28 - protein, 55 aa, Homo sapien
GSGMKETAAAKFERQHMDSPDLGTDDDDKAMAISDPNSHCYAGSGCPLLENVISK

SEQ ID NO: 29 - protein, 13 aa, Homo sapien
MKLLMVLMLAALS

SEQ ID NO: 30 - protein, 20 aa, Homo sapien
ALSQHCYAGSGCPLLENVIS

SEQ ID NO: 31 - protein, 20 aa, Homo sapien
GCPLLENVISKTINPQVSKT

SEQ ID NO: 32 - protein, 20 aa, Homo sapien
KTINPQVSKTEYKELLQEFI

SEQ ID NO: 33 - protein, 20 aa, Homo sapien
EYKELLQEFIDDNATTNAID

SEQ ID NO: 34 - protein, 20 aa, Homo sapien
DDNATTNAIDELKECFLNQT

SEQ ID NO: 35 - protein, 20 aa, Homo sapien
ELKECFLNQTDETLSNVEVF

SEQ ID NO: 36 - protein, 23 aa, Homo sapien
DETLSNVEVFMQLIYDSSLCDLF

SEQ ID NO: 37 - protein, 9 aa, Homo sapien
KLLMVLMLA

SEQ ID NO: 38 - protein, 9 aa, Homo sapien
LLMVLMLAA

SEQ ID NO: 39 - protein, 9 aa, Homo sapien
LMVLMLAAL

SEQ ID NO: 40 - protein, 9 aa, Homo sapien
FLNQTDETL

SEQ ID NO: 41 - protein, 10 aa, Homo sapien
LIYDSSLCDL

SEQ ID NO: 42 - protein, 10 aa, Homo sapien
KLLMVLMLAA

SEQ ID NO: 43 - protein, 10 aa, Homo sapien
FMQLIYDSSL

SEQ ID NO: 44 - protein, 10 aa, Homo sapien
AIDELKECFL

SEQ ID NO: 45 - protein, 10 aa, Homo sapien
LLQEFIDDNA

SEQ ID NO: 46 - DNA, 399 bp, Homo sapiens
atgcatcaccatcaccatcacacggccgcgtccgataacttccagctgtcccagggtgggcagggattcgccattccgatcgggcaggcgatggcgatcgcgggccagatcaagcttatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaa

SEQ ID NO: 47 - protein, 132 aa, Homo sapiens
MHHHHHHTAASDNFQLSQGGQGFAIPIGQAMAIAGQIKLMKLLMVLMLAALSQHCYAGSGCPLLENVISKTINPQVSKTEYKELLQEFIDDNATTNAIDELKECFLNQTDETLSNVEVFMQLIYDSSLCDLF

SEQ ID NO: 48 - DNA, 34 bp, Artificial Sequence
  PCR primer
gcgaagcttatgaagttgctgatggtcctcatgc

SEQ ID NO: 49 - DNA, 36 bp, Artificial Sequence
  PCR primer
cggctcgagttaaaataaatcacaaagactgctgtc

SEQ ID NO: 50 - protein, 9 aa, Homo sapiens
TLSNVECFM

SEQ ID NO: 51 - DNA, 309 bp, Homo sapiens
atgcagcatcaccaccatcaccaccacatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttga

SEQ ID NO: 52 - DNA, 306 bp, Homo sapiens
atgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttatttggcggccatcaccaccaccaccactaa

SEQ ID NO: 53 - DNA, 243 bp, Homo sapiens
atgcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttga

SEQ ID NO: 54 - DNA, 234 bp, Homo sapiens
atgggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttgatga

SEQ ID NO: 55 - DNA, 288 bp, Homo sapiens
atgcagcatcaccaccatcaccaccactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttatttggcggccatcaccatcaccatcactaa

SEQ ID NO: 56 - protein, 95 aa, Homo sapiens
MQHHHHHHHCYAGSGCPLLENVISKTINPQVSKTEYKELLQEFIDDNATTNAIDELKECFLNQTDETLSNVEVFMQLIYDSSLCDLFGGHHHHHH

SEQ ID NO: 57 - protein, 80 aa, Homo sapiens
MHCYAGSGCPLLENVISKTINPQVSKTEYKELLQEFIDDNATTNAIDELKECFLNQTDETLSNVEVFMQLIYDSSLCDLF

SEQ ID NO: 58 - protein, 101 aa, Homo sapiens
MKLLMVLMLAALSQHCYAGSGCPLLENVISKTINPQVSKTEYKELLQEFIDDNATTNAIDELKECFLNQTDETLSNVEVFMQLIYDSSLCDLFGGHHHHHH

SEQ ID NO: 59 - protein, 102 aa, Homo sapiens
MQHHHHHHHMKLLMVLMLAALSQHCYAGSGCPLLENVISKTINPQVSKTEYKELLQEFIDDNATTNAIDELKECFLNQTDETLSNVEVFMQLIYDSSLCDLF

SEQ ID NO: 60 - protein, 76 aa, Homo sapiens
MGSGCPLLENVISKTINPQVSKTEYKELLQEFIDDNATTNAIDELKECFLNQTDETLSNVEVFMQLIYDSSLCDLF

SEQ ID NO: 61 - DNA, 30 bp, Artificial Sequence
  PCR primer
cttgatcatatgaagttgctgatggtcctc

SEQ ID NO: 62 - DNA, 39 bp, Artificial Sequence
  PCR primer
ccgccgaattcatcaaaataaatcacaaagactgctgtc

SEQ ID NO: 63 - DNA, 32 bp, Artificial Sequence
  PCR primer
cacatgaagttgctgatggtcctcatgctggc

SEQ ID NO: 64 - DNA, 39 bp, Artificial Sequence; identical to SEQ ID NO: 62
  PCR primer

SEQ ID NO: 65 - DNA, 35 bp, Artificial Sequence
  PCR primer
cctattatcatatgggctctggctgccccttattg

SEQ ID NO: 66 - DNA, 34 bp, Artificial Sequence
  PCR primer
cctattatcatatgcactgctacgcaggctctgg

SEQ ID NO: 67 - DNA, 20 bp, Artificial Sequence
  PCR primer
cactgctacgcaggctctgg

SEQ ID NO: 68 - DNA, 23 bp, Artificial Sequence
  PCR primer
ggtgctcgagattatcagccgcc

SEQ ID NO: 69 - DNA, 503 bp, Homo sapiens
gacagcggcttccttgatccttgccacccgcgactgaacaccgacagcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttactacaaactacaagacaattgttgaaacctgctatacatgtttattttaataaattgatggca